(19) United States
(12) Patent Application Publication
(10) Pub. No.: US 2004/0235788 A1
(43) Pub. Date: Nov. 25, 2004

(54) Prebiotic compositions comprising fructans

(76) Inventors: Aliakbar Mobasseri, Voorhees, NJ (US); David Hakes, Willow Grove, PA (US); Kelly R. Fitzpatrick, Flourtown, PA (US)

Correspondence Address: Rachel Rondinelli, Neose Technologies, Inc., 102 Witmer Road, Horsham, PA, 19044, US

(21) Appl. No.: 10/817,491

(22) Filed: Apr. 2, 2004

Related U.S. Application Data
(60) Provisional application No. 60/460,754, filed on Apr. 4, 2003.

(51) Int. Cl. A61K 31/715

(52) U.S. Cl. 514/54

(57) ABSTRACT

Fructan compositions for stimulating the growth of bifidogenic bacteria and/or other beneficial bacteria including compositions comprising one or more branched or unbranched levan, compositions comprising one or more branched FOS, and compositions comprising mixtures of fructans of a single type but different degree of polymerization. These compositions include compositions comprising short-chain fructans and compositions predominately comprising glucose-terminated fructans and are disclosed as food additives, sweeteners, and bulking agents.

### BACKGROUND OF THE INVENTION

1. Field of the Invention

The present invention relates to compositions comprising polyfructans which promote microbial balance in humans and animals.

2. Discussion of the Background

Certain intestinal bacteria such as bifidobacteria are generally considered beneficial for humans and animals whereas other intestinal bacteria such as E. coli, many enterobacteria and clostridia, are generally considered unfavorable for humans and animals, either due to invasive action or the production of toxic metabolites. See, Balows et al., 1991, Manual of Clinical Microbiology, 5th edition, Washington, ASM Press. Fructooligosaccharides (“FOS”) found in certain foods are thought to promote the growth of bifidobacteria over pathogenic bacteria. FOS are not completely digested but travel to the colon where they are selectively fermented by bifidobacteria into lactate and short-chain fatty acids. Thus, as an energy source for bifidobacteria and not certain pathogenic bacteria, FOS can selectively promote the growth of favorable bacteria. In addition, by enhancing the production of short-chain fatty acids, FOS can help create an intestinal environment that does not favor the growth of some pathogenic bacteria.

FOS occur naturally in foods such as onions, bananas, tomatoes, honey, barley, garlic, chicory, Jerusalem artichoke and wheat. Designing a diet encompassing those foods for obtaining the benefits of FOS is difficult because of the low concentration of FOS in most foods. Further, it can be difficult or expensive to extract and process FOS from those natural sources industrially. The FOS in those sources, for example in Jerusalem artichoke, can also have drawbacks such as production of excessive gas (which is dependent upon FOS molecular weight) high caloric content, and inconsistent preparations. The material extracted from chicory and Jerusalem artichokes, for example, includes distributions of polymers that vary with the plant source, the planting location, and the harvest time.

Raftilose and Neosugar are commercial FOS products. Raftilose is a mixture of FOS ranging from two to seven monosaccharides in length, averaging four sugar moieties, and is produced by hydrolysis of inulin extracted from chicory. Raftilose therefore predominately comprised of fructosyl chains which do not have terminal glucose. Neosugar is 30% by weight 1-kestose (GF₂), 57% by weight 1-nystose (GF₃), and 13% by weight 1^{F}-fructosylnystose (GF₄). Neosugar is produced from sucrose by the action of fungal fructosyltransferase and therefore predominantly consists of fructosyl chains which do not have terminal glucose units.

Accordingly, there remains a need for polyfructan compositions which can promote the growth of bifidobacteria yet avoids the drawback of producing excessive gas.

### SUMMARY OF THE INVENTION

In one aspect, the present invention relates to compositions comprising at least one fructan, the total fructan content being present in a prebiotic amount.

The composition comprises short-chained unbranched fructans having an average degree of polymerization (“DP”) of 4. In a preferred embodiment, the composition comprises unbranched FOS, wherein the unbranched FOS predominately have terminal glucose units. The composition may comprise mixtures of fructans, either mixtures of a single type of fructan-or mixtures of different types of fructan. The composition may comprise at least one fructan chosen from branched and unbranched levans. The composition may comprise at least one fructan chosen from branched FOS.

Another embodiment of the present invention relates to methods of promoting internal microbial balance. The method comprises administering a composition comprising an indigestible or partly indigestible fructan or mixture of fructans to a human or animal in an amount effective for promoting the growth of beneficial intestinal bacteria, for example bifidobacteria and lactobacilli. Administration is via a means which will result in delivery of the fructan to the intestinal region of the human or animal. The method may also comprise applying a cream, for example a vaginal cream, comprising one or more fructans in an amount effective for promoting the proliferation of beneficial bacteria.

In another embodiment, the growth of one or more beneficial bacteria is selectively promoted over the growth of pathogenic bacteria. The fructan composition can be administered orally per se, or can be incorporated into food and/or drink products, for example to promote intestinal microbial balance. The fructan composition can be applied as a cream, for example to promote vaginal microbial balance.

### BRIEF DESCRIPTION OF THE DRAWINGS

A more complete appreciation of the invention and many of the attendant advantages thereof will be readily obtained as the same become better understood by reference to the following detailed description when considered in connection with the accompanying drawings, wherein:

FIG. 1 is a chart reporting results of an experiment for evaluating the growth of Lactobacillus acidophilus 4357, a beneficial bacteria, in MRS media with and without sugars.

FIG. 2 is a chart reporting results of an experiment for evaluating the growth of Lactobacillus johnsonii 33200, a beneficial bacteria, in MRS media with and without sugars.

FIG. 3 is a chart reporting results of an experiment for evaluating the growth of Lactobacillus amylovorus 33620, a beneficial bacteria, in MRS media with and without sugars.

FIG. 4 is a chart reporting results of an experiment for evaluating the growth of Lactobacillus amylovorus 33198, a beneficial bacteria, in MRS media with and without sugars.

FIG. 5 is a chart reporting results of an experiment for evaluating the growth of Lactobacillus johnsonii, a beneficial bacteria, in MRS media with and without sugars.

FIG. 6 is a chart reporting results of an experiment for evaluating the growth of Lactobacillus plantarum, a beneficial bacteria, in MRS media with and without sugars.

FIG. 7 is a chart reporting results of an experiment for evaluating the growth of E. coli 23502 (05-k4) a pathogenic bacteria, in MRS media with different sugars.

FIG. 8 is a chart reporting results of an experiment for evaluating the growth of E. coli 41-41 (05-k4) a pathogenic bacteria, in MRS media with different sugars.

FIG. 9 is a chart reporting results of an experiment for evaluating the growth of E. coli 01578:H7 (021901-1) a pathogenic bacteria, in MRS media with different sugars.

FIG. 10 is a chart reporting results of an experiment for evaluating the growth of Samonella group B (021901-5) a pathogenic bacteria, in MRS media with different sugars.

FIG. 11 is a chart reporting results from rat ADME studies.

FIG. 12 is a chart providing the DP distribution of a synthetically produced fructan having DP2-DP29 and a mode of DP29-determined by size exclusion/light scattering 4010LI40.

FIG. 13 is a chart providing the DP distribution of a synthetically produced short-branched fructan having DP2-DP14, and a mode of DP6 4030LI40.

FIG. 14 is a chart providing the DP distribution-of a synthetically produced fructan having DP3-DP8 and a mode of DP4.

FIG. 15 is a chart showing size-exclusion/light-scattering analysis of shortbranched fructan 4030L140.

FIG. 16 is a chart showing size-exclusion/light-scattering analysis of levan 4020L140.

FIG. 17 is a chart providing the DP distribution of two levan preparations analyzed by high-performance anion exchange chromatography with pulse amperometric detection (HPAEC-PAD).

FIG. 18 is a chart providing the DP distribution of two branched fructan preparations analyzed ;by high performance anion exchange chromatography with pulse amperometric detection (HPAEC-PAD)

FIGS. 19-23 illustrate the fructan metabolism for various Lactobacillus bacteria.

FIGS. 24-25 illustrate the sugar metabolism of Eschericia coli.

FIGS. 26-27 illustrates the FOS metabolism of pathogenic bacteria.

FIGS. 28-44 further illustrate aspects of the present invention.

### DETAILED DESCRIPTION OF THE PREFERRED EMBODIMENTS

The inventors have discovered heretofore undisclosed fructan compositions for stimulating the growth of bifidogenic bacteria and/or other beneficial bacteria. Those compositions include compositions comprising: one or more branched or unbranched levan; one or more branched or unbranched inulin or FOS; and compositions comprising mixtures of fructans of a single type but different degrees of polymerization, or of different types, or both. The inventors have also discovered compositions with improved results for promoting the growth of bifidobacteria, and promoting internal microbial balance, over known compositions. Those compositions include compositions comprising short-chain fructans and compositions predominately comprising glucose-terminated fructans (i.e. predominately comprising GFn rather than Fn compounds). The composition is not solely a mixtuer of 1-kestose, 1-nystose, and 1^{f}-fructosylnystose.

### 5.1. DEFINITIONS

Within the context of the present invention, the term “fructan” is understood to encompass inulins and levans, both branched and unbranched, and therefore refers to branched and unbranched oligosaccharides and polysaccharides formed from monomers of fructose furanosyl rings, optionally having a glucose moiety attached to the reducing end of the oligo- or polysaccharide. Fructans derived entirely from fructose monomers are represented by the shorthand “Fn,” where F is a fructose unit and n is the number of such units. Such fructans are typically the breakdown products obtained from larger fructans by hydrolysis. Fructans including a terminal glucose are represented by the shorthand “GFn,” where G is a glucose unit and F and n are defined as above. Such fructans include natural levans or inulins.

The term “inulin” refers to one of the two main types of fructans. Inulins comprise predominantly β-(2→1)-D-fructofuranosyl units. Because inulins and levans are synthesized by sequential transfer of a fructosyl units onto an initial sucrose acceptor molecule, both fructans have a terminal glucosyl group in their unhydrolyzed states. Inulins may be branched or unbranched. Unlike inulins and levans synthesized in vitro using purified enzymes, naturally derived inulins and levans may exhibit differing degrees of branching. Thus, it is to be understood that within the context of the present invention, the terms “inulin” and “levan” encompass, in addition to fully unbranched species, species having a varying degree of branching, such as for example species having less than about 5% sugars that have both β-(2→1) and β-(2→6) linkages (Tables 2 and 3). Levans comprise predominantly β-(2→6)-D-fructofuranosyl units. Like inulins, under the foregoing definitions, levans may also be branched or unbranched, such as a β2,6-linked backbone, branched with β2,1-linked units.

“Branched fructans” refers to levans or inulins having a branched fructose furanosyl structure. In the case of inulins, for which the main chain consists of β-(2→1)-D-fructofuranosyl units, branching is due to β-(2→6)-D-fructofuranosyl linkages to the main fructan chain. Conversely, in the case of levans, for which the main chain consists of β-(2→6)-D-fructofuranosyl units, branching is due to β-(2→1)-D-fructofuranosyl linkages to the main fructan chain. The degree of branching affects both physicochemical properties such as viscosity and bulking ability, and also biological properties such as suitability as carbon sources to support the growth of different bacterial strains, as set forth below. Branched levans and inulins are defined as having about 5% or more sugars that have both β-(2→1) and β-(2→6) linkages (see, for example, Tables 2 and 3). It is further to be understood that where a fructan structure is stated with sufficient specificity to determine whether that species is or is not chemically branched, considerations of whether that fructan is or is not predominately branched do not apply.

The degree of polymerization (DP) of a fructan is the total number of combined fructose and glucose units of a particular fructan. Thus, for the prototypical inulin, 1-kestose, and the prototypical levan, 6-kestose, the DP is 3 (herein, DP3). In natural plant levans and inulins, the DP may be as high as several hundred, while bacterial and fungal levans and inulins may have a much higher DP of greater than 100,000. The DP includes all fructose and glucose units within a fructan, irrespective of whether a unit is part of a branched portion of a fructan or a part of the main fructan chain.

Due to current limitations associated with manufacturing fructans, it should be understood that, unless stated otherwise, preparations of fructans typically encompass a distribution of fructans of varying DP rather than a single DP. Therefore, compositions of “a fructan” “an inulin,” and “a levan” can respectively include a range of fructans of varying DP, a range of inulins of varying DP, or a range of levans of varying DP, rather than a single DP. Consequently, the phrases “a fructan,” “an inulin,” and “a levan” can refer to the mode in a distribution of fructans of varying DP. The mode is the value (DP) that occurs most frequently on a molar basis in the distribution.

Likewise, the phrase “a fructan of DPn,” unless otherwise stated, can refer to distributions of fructans, where the mode of the distribution of degrees of polymerization corresponds to n. Likewise, the phrase “an inulin of DPn,” unless otherwise stated, can refer to distributions of inulins, where the mode of the distribution corresponds to inulins of degree of polymerization n, and the phrase “a levan of DPn,” unless otherwise stated, can refer to distributions of levans, where the mode of the distribution corresponds to levans of degree of polymerization n. Thus, where a specific fructan species is indicated, such as an FOS having DP3, it is understood that the DP3 FOS can be a distribution of FOS, the mode of the distribution corresponding to an FOS of degree of polymerization 3.

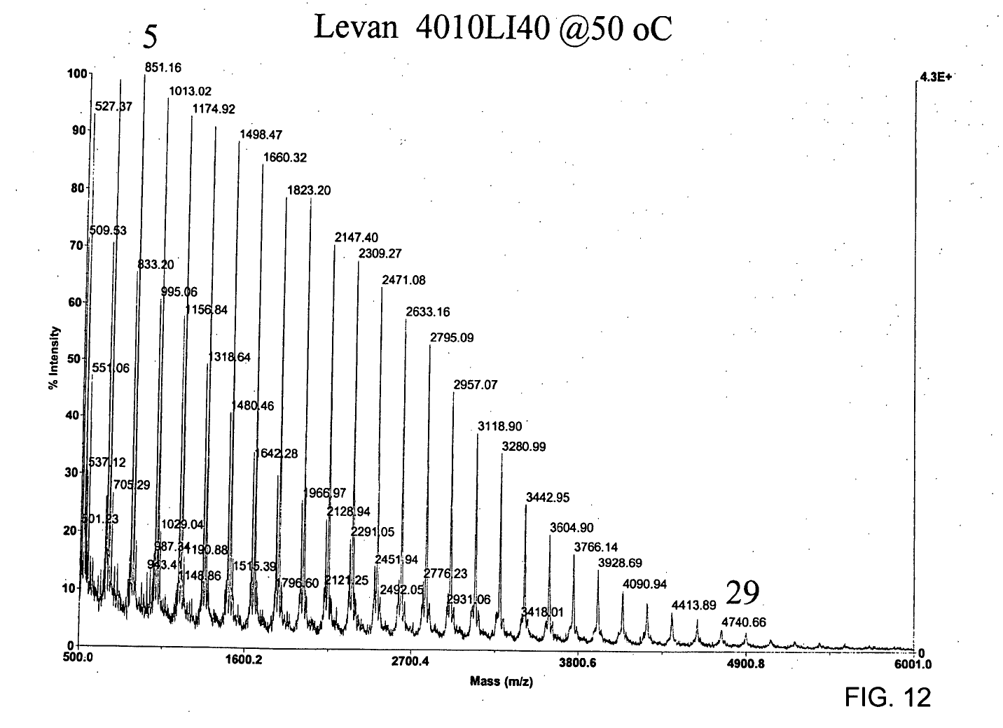

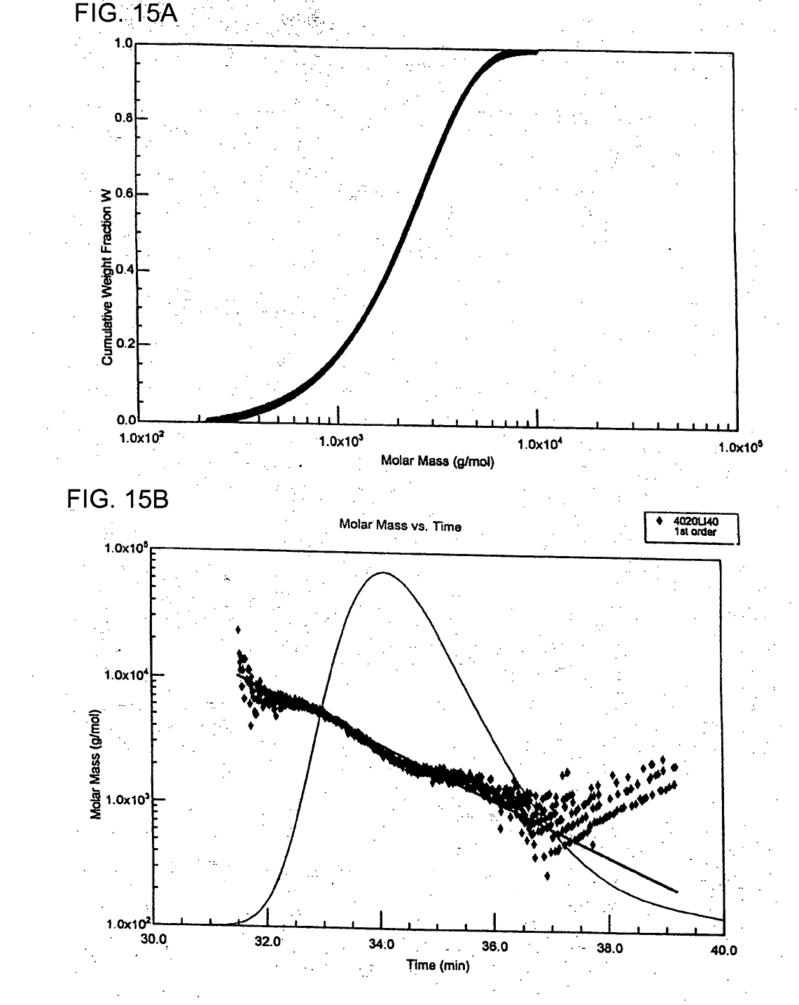

FIGS. 12-15 exemplify these definitions, i.e. fructans which include a distribution of DP. As discussed above, these fructans are referred to by a specific DPn corresponding to the mode of the distribution. For example, FIG. 15 shows a matrix-assisted laser desorbtion/ionization time-of-flight spectrum for a preparation of fructan of DP4. This particular fructan is identified herein as preparation FOS, or “high FF(FOS)”. Referring to FIG. 12, again by way of example, the ion which corresponds to a m/z ratio of 851.2 is assigned as a sodium adduct of DP5. It is further apparent that the sample comprises a distribution of fructans ranging from about DP2 to about DP29.

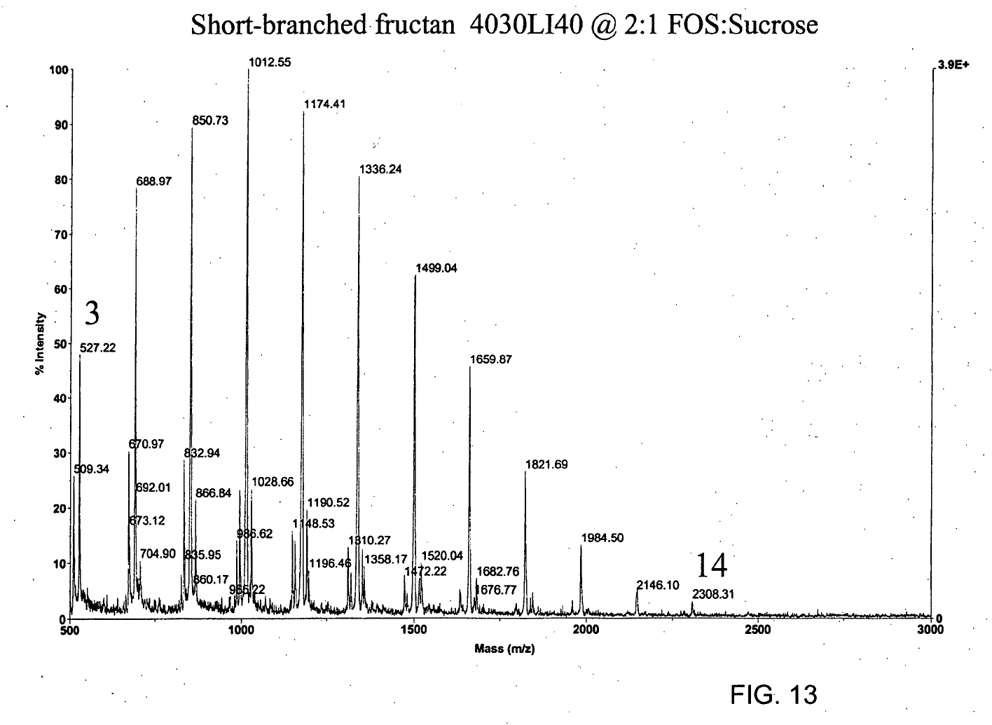

FIG. 13 illustrates a fructan of mode DP6, and demonstrates that this fructan is actually a distribution of fructans having DP ranging from about 2 to about 14. FIG. 12 illustrates a fructan of mode DP5, and also demonstrates that this fructan is actually a distribution of fructans having DP ranging-from about 3 to about 29.

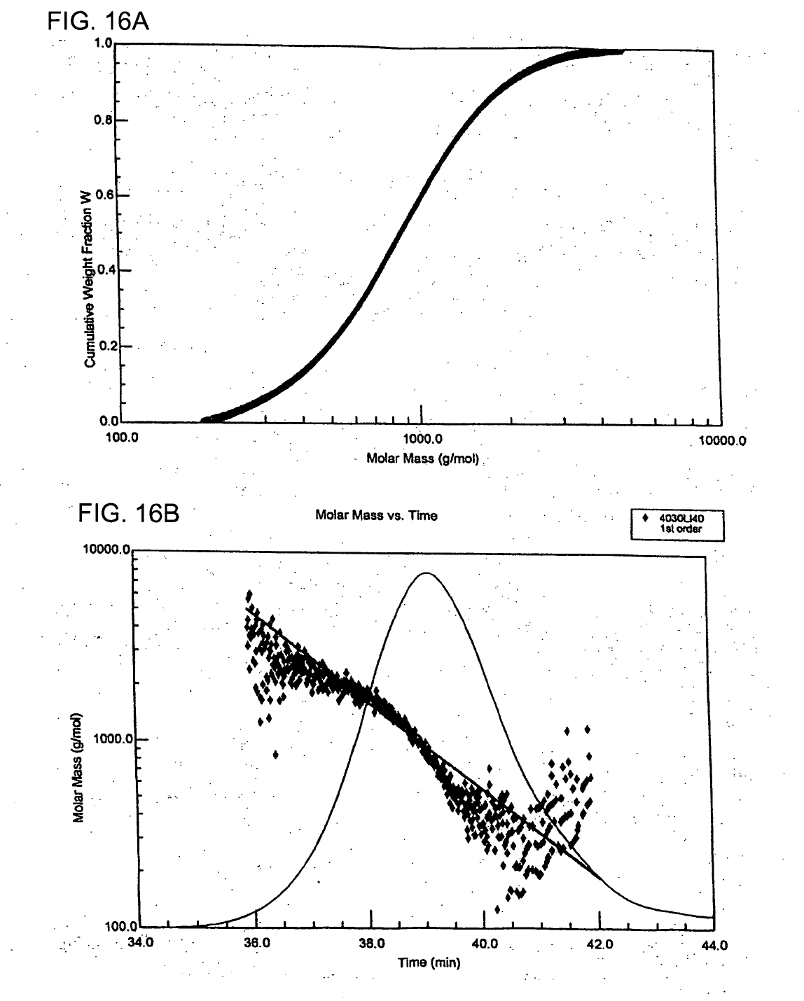

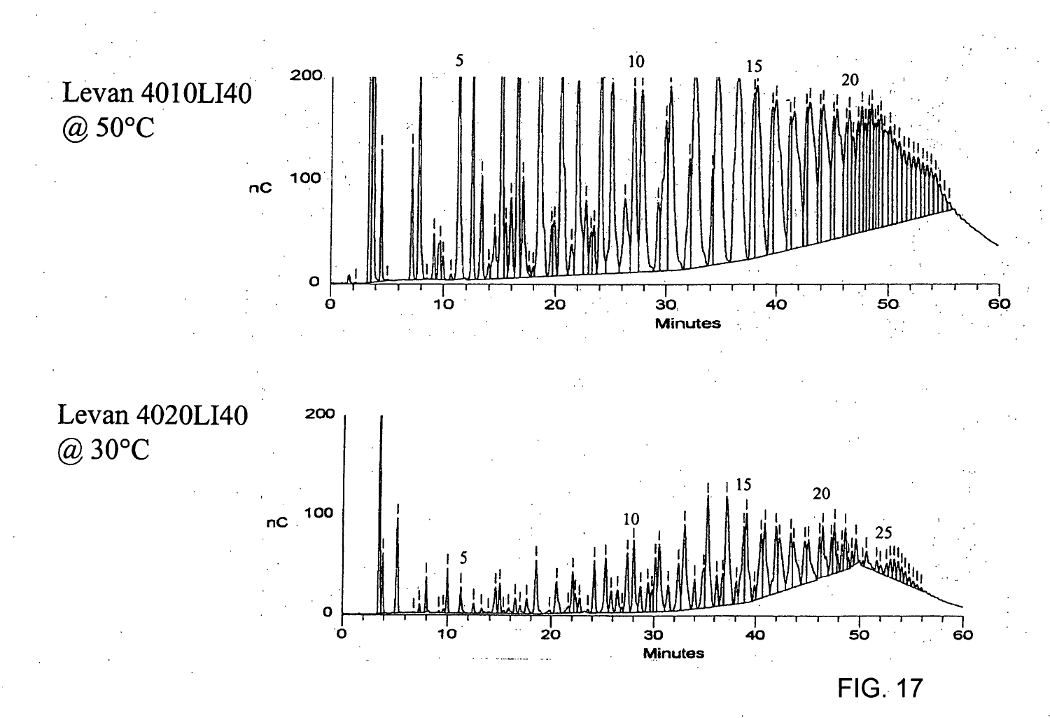

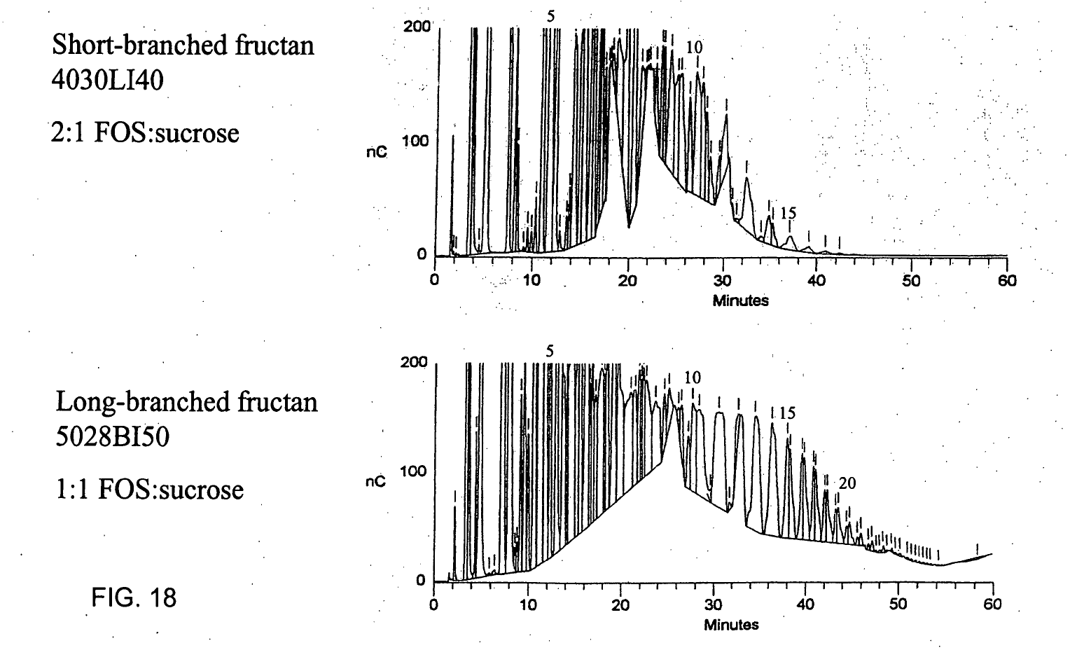

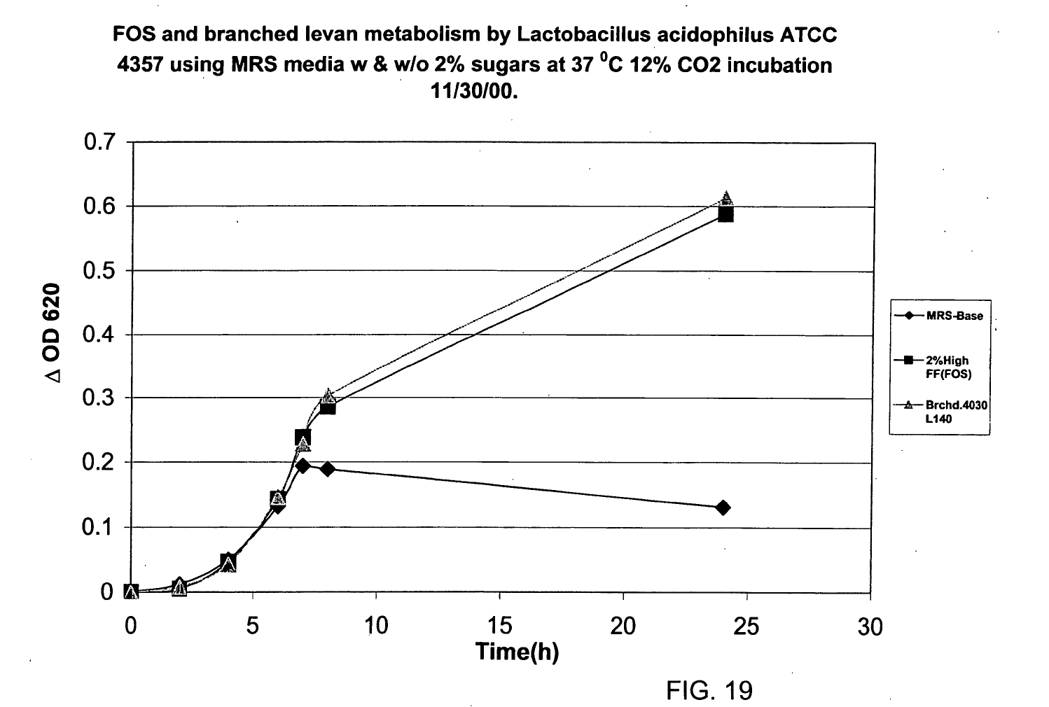

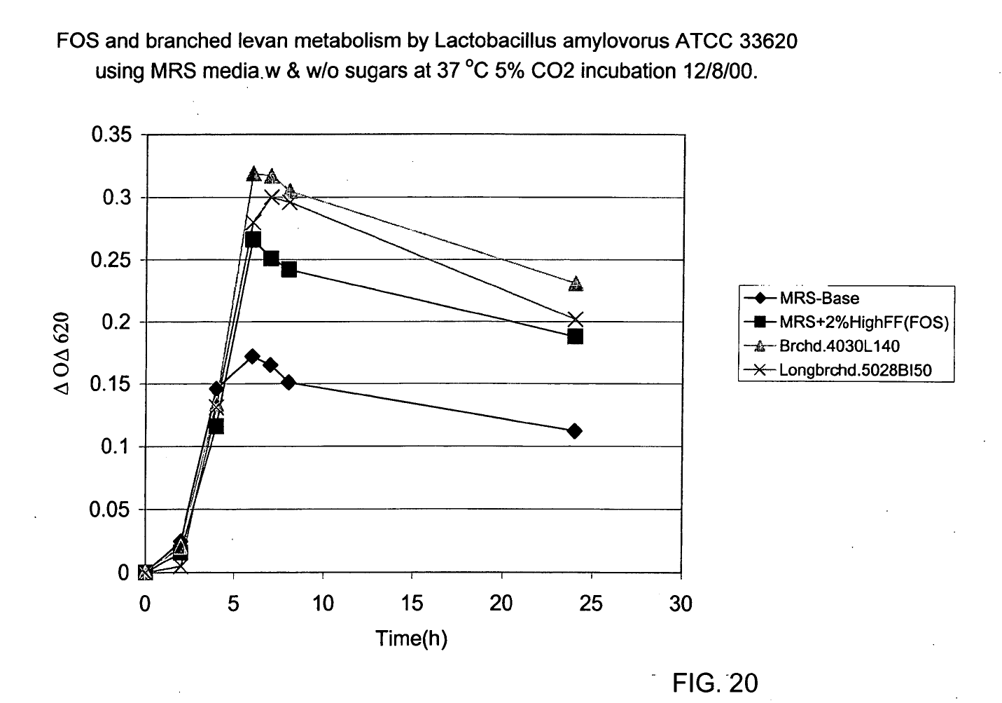

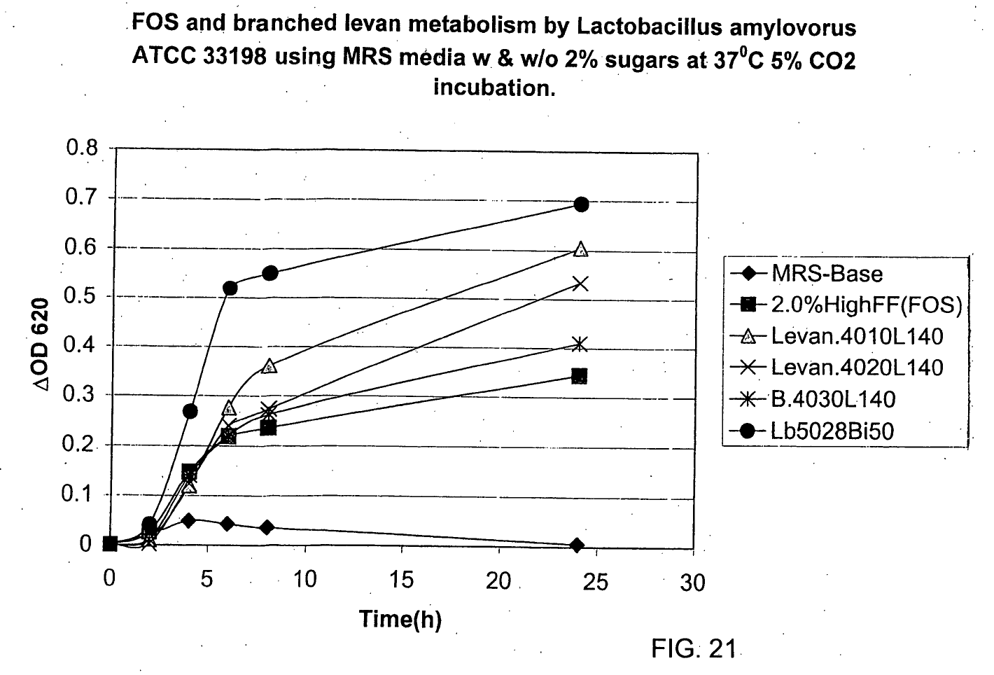

In certain embodiments, an alternative technique for measuring the molar abundance of fructan species and assessing DP may be preferred. MALDI-TOF spectra are provided herein to illustrate the distribution of degrees of polymerization in fructan preparations and thereby provide one working definition for DP, but may not always provide an accurate measurement of the mode of DP when relying upon peak height to represent molar abundance. Therefore, other measures of DP can also be used. A quantitative measure of the mode DP is obtained, for example, by size-exclusion/light-scatter analysis (FIGS. 15 and 16), as described below (section 5.2.3), or by HPLC analysis. A qualitative measure of the mode DP may be obtained by Dionex chromatography, as illustrated in FIGS. 17 and 18.

The term “mixture” as applied herein to fructans denotes the combination of two or more preparations of fructan, rather than the distribution of degrees of polymerization typically present within each fructan preparation unless otherwise noted. Thus, as indicated above, where a certain DP is indicated, i.e. a specific fructan species is indicated, for example an FOS having DP3, it is understood that the DP3 FOS is actually a distribution of FOSs. Therefore, in accordance with the definition of “mixture,” a mixture of fructans, for example a mixture of fructans of DP3 and fructans of DP8, encompasses a combination of two distributions of fructans, wherein the mode of one distribution is DP3 and the mode of a second distribution is DP8, and the mode(s) of the mixture must lie within the range 3-8, including endpoints.

The term “predominately GFn,” for example when used in the phrase a fructan having a DPn and being predominately GFn, is meant to account for impurities that can occur when purifying (including synthesizing) fructans. In other words, for example, while a fructan is produced according to a synthetic method which should obtain an amount of fructan having specific DPn, and each fructan in that amount should have a terminal glucose, it may be possible that some of the fructans are Fn. Likewise, the amount of fructan contains a distribution of amounts of fructans with varying DPn, all of which are intended to be GFn, but may include some Fn impurity.

The term “predominately Fn” is intended to account for the fact that although fructans produced by hydrolysis have a large majority of Fn units, they may also include GFn impurity.

The term “fructooligosaccharide,” unless otherwise noted, means any fructan in the subset of inulins of generic formula GFn, where n ranges from 2 to 9. As used herein, “FOS” is the abbreviation for both fructooligosaccharide and fructooligosaccharides.

It will be appreciated that the proportion of GFn to Fn in a particular preparation of fructans can depend on how the fructan is prepared. FOS, for example, occur naturally in many plant species such as onion, edible burdock, asparagus, and wheat. However, FOS can also be prepared from sucrose by the transfructosylating action of enzymes as detailed below. These natural and synthetic preparations typically contain predominantly GFn because they are synthesized by sequential addition of fructose to a sucrose acceptor. Alternatively, FOS are prepared by limited hydrolysis of longer chain inulins. When prepared by hydrolysis, such FOS contain a higher proportion of Fn compared to GFn.

The term “short-chain” branched fructan refers to fructans having chain lengths that correspond to the length of chains of synthetically-prepared fructans, wherein the method of synthesis is according to processes described in U.S. Pat. Nos. 5,998,177 and 5,925,205, which are herein incorporated by reference, processes described herein, and other processes in which at least fructooligosaccharides and sucrose are used as starting materials, and the ratio of FOS to short-chain branched fructan ranges from about 1.5:1.0 to about 5.0:1.0. Fructans corresponding in chain length to those produced by these methods, but produced by a different method are also considered short chain fructan. In this embodiment, the ratio is about 1:0.5. The term “short-chain” branched fructan can also encompass any fructan having a DP ranging from about 3 to about 8. FIG. 13 exemplifes a short-chain fructan.

The term “long-chain” branched fructan refers to fructans having chain lengths corresponding to the length of chains of synthetically-prepared fructans, wherein the method of synthesis is according to processes described in U.S. Pat. Nos. 5,998,177 and 5,925,205, processes described herein, and other processes in which at least fructooligosaccharides and sucrose are used as starting materials, and the ratio of FOS to sucrose ranges from about 0.5:1.0 to about 1.0:1.0. In this embodiment, the ratio is about 1:1. Fructans corresponding in chain length to those produced by these methods, but produced by a different method are also considered long chain fructan. The term “long-chain” branched fructan can also encompass any fructan having a DP that includes a significant amount of fructan having a DP of greater than about 9.

The term “prebiotic amount” means an amount of fructan (total fructan, if the fructan is a mixture) which contributes to microbial balance within an internal body cavity that has flora in a healthy human, such as, for example, the intestine or vagina. In some embodiments a prebiotic amount stimulates the growth of bifidogenic bacteria, while having little impact or suppressing the growth of pathogenic strains of E. coli or salmonella. For example a prebiotic amount is would be an amount sufficient to promote bifidogenic bacteria over pathogenic bacteria at a rate of at least 1.2:1, preferably at least 2:1, more preferably at least 3:1, more preferably at least 5:1, more preferably at least 10:1, more preferably at least 50:1 and even more preferably at least 100:1. Those of skill in the art would be able to determine whether a composition contained a prebiotic amount of fructan. For example, a prebiotic amount for a human could be between about 1 and about 100 g of fructan per day, or between about 3 and about 15 g of fructan per day The term “bifidogenic bacteria” herein includes bacteria of the genus Bifidobacterium, which consists of at least 25 distinct species including, but not limited to, B. adolescentis, B. longum, B. breve, B. inventis, and B. bifidum. The growth of bifidogenic bacteria and other beneficial colonic and vaginal bacteria, such as lactobacteria, including L. acidophilus and L. johnsonii are also enhanced by the compositions of the present invention. The large intestine is the most heavily colonized region of the human digestive tract, and the composition of this microflora have important consequences for human health. See, Draser, B. S. & Roberts, A. K. (1989), “Control of the large bowel microflora,” in Hill, M. J. & Marsh, P. D. (Eds.): Human Microbial Microbial Ecology, Boca Raton, Fla. CRC Press Inc., 1989, pp.87-110.

As used herein, the term “fructosyltransferase” refers to any enzyme or enzymes capable of transferring a fructose moiety from sucrose as a donor to another saccharide (e.g. a fructan). In preferred embodiments, the enzyme selectively transfers the furanose form of fructose from sucrose or fructans to produce β-(2→1)-D-fructofuranosyl or β-(2→6)-D-fructofuranosyl linkages. The β-2,1-fructosyltransferase may transfer fructose to the C₁ position of terminal fructose, thereby forming a linear chain extension. The β-2,6-fructosyltransferase may transfer fructose to the C₆ position of terminal fructose, thereby also forming a linear chain extension. Where branched fructans are desired, β-2,1-fructosyltransferase may be combined with β-2,6-fructosyltransferase in proportions appropriate to the desired degree of branching, as described in U.S. Pat. Nos. 5,998,177, and 5,925,205. It will be apparent that fructans prepared enzymatically have, as their initial acceptor, sucrose, and therefore have a glucose at the reducing terminus. Such fructans are denoted GFn. It will be further appreciated that, in the absence of a hydrolysis step, the content of fructooligosaccharides (Fn) will be lower than that contained in preparations having the same distribution of DP, but prepared by hydrolysis.

The term “purified” in the present invention broadly encompasses fructans, distributions of fructans, and mixtures of fructans. The term “purified” does not require that the fructan(s) be isolated from all other compounds, but merely that they are distinct from the fructans of the natural source either because they have been further separated from the natural source or because they have been synthetically prepared. As discussed above, due to limitations with current methods of synthetically producing fructans, a fructan of, for example, DP10 may actually include a distribution of fructans of varying DPs. The synthetically-produced fructan having DP10 (and being in fact a distribution of fructans of varying DP, wherein the mode DP is 10) would be considered herein a “purified” fructan.

“Purified” fructans would also include fructan preparations that are treated, for example, in the following manner to substantially remove non-fructan-components: Following enzymatic treatment of a solution comprising sucrose the product can be separated from high molecular weight compounds using 10K ultrafiltration, the product can be decolorized using a resin treatment step with, for example, FDA-approved resins such as Amberlite IRA958C1 or XAD16HP; substantially separated from simple sugars such as glucose, fructose, and sucrose by membrane filtration, such as for example by a spiral wound nanofiltration membrane; and, optionally, spray dried. “Purified” fructan can optionally be subjected to further steps to narrow the distribution of the degrees of polymerization of the fructans present in the composition. For example, fractionation by size can be achieved by any of a number of size-fractionation techniques known in the art, including but not limited to size-exclusion chromatography, selective alcohol precipitation, membrane ultrafiltration, and the like. By these means, purified fructan preparations having a narrower distribution of DP about a characteristic mode DP compared to prior art preparations can be obtained which are largely free of simple sugars and fructooligosaccharides. For example, spiral wound nanofiltration membranes can be expected to remove simple sugars from a fructan preparation to an amount less than about 5% by weight. As shown below, such purified fructans have advantageous properties.

Relative terms, e.g. “about” and “substantially,” are used in this specification to account for imprecisions that can occur in the methods and measurements involved in preparing compounds and compositions according to this invention. Thus, for example, by “substantially” removing non-fructan components from the fructans, it is meant that while the purpose of the preparation method chosen may be to remove all non-fructan components, limitations in the methods may result in some impurities remaining in the fructan preparation.

### 5.2 METHODS OF SYNTHESIZING AND OBTAINING FRUCTANS ACCORDING TO THE INVENTION

Fructans according to the present invention can be synthesized or obtained according to processes known in the art. For example, fructans can be extracted from natural sources by methods known in the art, such as, for example, hot-water extraction. Alternatively, fructans, for example, fructooligosaccharides, can by prepared by enzymatic hydrolysis using inulinase according to methods known the art. The fructan compositions according to the present invention can also comprise fructans prepared enzymatically using fructosyltransferases as described in U.S. Pat. No. 5,998,177 and U.S. Pat. No. 5,952,205.

Having generally described this invention, a further understanding can be obtained by reference to certain specific examples which are provided herein for purposes of illustration only and are not intended to be limiting unless otherwise specified

### I. Process Description

Levans are prepared in an enzymatic reaction that uses sucrose as the starting material.

A mixture comprising levans of varying saccharide units (DP 3 to about 20 or more), glucose, fructose, difructose, and sucrose is synthesized by the action of a levan sucrase (e.g. from B. subtilis) on sucrose. The enzyme is a purified recombinant product expressed in E. coli JM109, or is obtained non-recombinantly from, for example, B. subtilis in an impure (lysate) or purified state.

The levan mixture and the simple sugars (fructose, sucrose, glucose, and difructose) are separated from protein using, for example, hollow fiber (HF) membranes. Separation of the simple caloric low molecular weight sugars from the levans is achieved, for example, by nanofiltration on spiral wound membranes. The levans are rendered colorless and odorless by resin treatment. The clear, colorless levan solution is then spray-dried to give a white powdered product.

All materials used throughout the process are stainless steel, polypropylene, or high-density polyethylene.

### II. Production of an Exemplary Levansucrase by E. coli JM109 Cells

In the present embodiment, the levansucrase (SacB) gene is cloned from DNA isolated from B. subtilis (ATCC 6051) using standard molecular techniques. The levansucrase gene is ligated into pGex Kan^{R} vector and transformed into E. coli JM109 strain, which is obtained from the ATCC. An original transformant culture is grown in LB broth containing kanamycin until mid-exponential growth phase and then aseptically transferred into a bottle containing sterile cold LB-glycerol medium. The culture-LB-glycerol mixture is distributed into 100×400-500 μL aliquots into sterile Nalgene™ cryovials. These vials are stored in liquid nitrogen. Samples are visually inspected for integrity and tested for homogeneity, phage and viability.

A Master Cell culture and working cell cultures are grown in LB broth until mid-exponential growth phase and then aseptically transferred into a bottle containing sterile cold LB-glycerol medium. The culture-LB-glycerol mixture was distributed into 100×400-500 μL aliquots into sterile Nalgene™ cryovials. These vials are stored in liquid nitrogen. Samples are visually inspected for integrity and tested for homogeneity, phage and viability.

Other enzymes can also be used in lieu of or in addition to levansucrase obtained from the SacB gene. Non-limiting examples of suitable fructosyltransferases may be obtained from microorganisms of the genus Aspergillus such as A. oryzae ATCC 20498; A. sp. ATCC 20524; A. awamori, A. sydowi and A. niger ATCC 20611 from the genus Penicillium such as P. jancezewskii ATCC 10115 and 26546; P. nigricans, from the genus Fusarium such as F. lini IAM 5011; and from the genus Aureobasidium such as A. pullulans ACTT 9348; Streptococcus mutans ATCC 25175; and A. pullulans var. melanigenum A-8 ATCC 20612. Suitable enzymes may also be obtained from yeasts and other microorganisms such as: the genus Saccharomyces, such as S. cerevisiae; the genus Rhodotorula such as R. lutinis; the genus Pichia such as P. miso; the genus Hansenula such as H. miso; the genus Candida such as C. tropicalis; and from higher plants such as asparagus, dahlia tubers, chicory roots and the Jerusalem artichoke as described in JP-A-56-154967 and JP-B-59-53834. Another fructosyltransferase (also known as a levan synthetase) having a β-2,6 linkage forming activity, may also be used. A combination of fructosyltransferases having both β-2,1 and β-2,6 linkage forming activity may be used together, to form either a polyfructan having a homogenous distribution of β-2,1 and β-2,6 linkages or blocks comprising β-2,1 linkages and blocks comprising β-2,6 linkages.

A preferred enzyme is a bacterial fructosyltransferase which is obtained and expressed from a gene isolated from Streptococcus mutans. In particular, S. mutans ATCC 25175 is a source of a fructosyltransferase gene. The fructosyltransferase is obtained as a fusion construct with a heterologous protein sequence. A suitable fusion protein is, for example, the fructosyltransferase isolated from Streptococcus mutans fused to the C-terminal of glutathione-S-transferase.

The coding sequence of the Streptococcus mutans frucotosyltransferase, lacking the predicted signal sequence is isolated from Streptococcus mutans strain ATCC 25175 by PCR which is used to form a transformant that expresses a fructosyltransferase fusion protein. Another suitable fructosyltransferase gene sequence from Streptococcus mutans strain GS-5 is reported by Shiroza, T. and Kuramitsu, H. K. J. Bacteriol., 170, 810-816 (1988).

In some embodiments, the fructosyltransferase is purified. As used herein with reference to enzymes the term “purified” means that the enzyme has been purified, at least partially, from the host organism in which it is produced, in order to effect the at least partial removal of degradative enzymes which would otherwise degrade the fructan and/or proteases which would otherwise degrade the fructosyltransferase enzyme. The enzyme can also be purified to a degree such that there is the practical absence of degradative enzymes. When the source of the enzyme is a transfected E. coli microorganism, a crude cell lysate may be used, if the transfected E. coli has little or no native fructosyltransferase of fructan degrading enzymes.

### A. Fermentation Process

A sample of the E coli JM109 Kan^{R} Levansucrase working cell culture is aseptically transferred to LB medium containing kanamycin and dextrose. The culture is grown at 37° C. with agitation until exponential growth phase where an aliquot is aseptically transferred to a fresh sterile LB medium containing kanamycin and dextrose. The culture is allowed to grow overnight at 37° C. with agitation, which corresponds to exponential growth phase. The E coli seed culture is transferred to a fermenter containing sterile production medium comprising the ingredients listed in Table 1, or any other suitable medium known in the art for the growth of E. coli.

TABLE 1
E coli JM109 Kan^{R} Levansucrase Exemplary
Production Medium
Ingredients
Yeast Extract
Tryptone
Dextrose
Kanamycin Sulfate
Potassium Phosphate
Ammonium Phosphate
Magnesium Sulfate
Polyglycol P-2000 (antifoam)

The fermenter monitors and controls the system physical parameters including pH, temperature, dissolved oxygen, agitation, aeration, nutrient addition and foaming. The pH is controlled by addition of sodium hydroxide or suitable choice of acid. The fermenter is sterilized and cleaned-in-place prior to every use. A sample of the medium is taken prior to inoculation of the fermenter and is tested for sterility. Expression of levansucrase is induced by the addition of IPTG, Isopropyl-β-D-thiogalactopyranoside, or lactose, during the exponential growth phase of the E coli culture as determined by turbidity.

### B. Harvest and Lysis of E. coli

A sample of the culture is tested for homogeneity. The E coli culture is chilled to <10° C. in the fermenter. The culture is harvested and subjected to high-speed centrifugation to sediment the cells and to remove the medium supernatant, which is discarded. The cells are resuspended in chilled reverse osmosis purified water and the suspension is homogenized at 8000-10,000 psi. The lysate is clarified by centrifugation and the cell debris is discarded. The clarified lysate is tested for levansucrase activity and is stored at −40° C.

### H. Synthesis

The sucrose input is supplied as refined sugar crystals, crude sugar syrup, molasses, corn syrup or equivalent sucrose source. The sucrose is added to 30-35° C. water in a stainless steel mix tank. The sucrose solution is pumped into the reactor through a 0.2 μm polypropylene filter.

The reactor is a stainless steel closed tank with an insulating water jacket to allow for temperature control during operation. The pressure in the headspace is equilibrated with the atmosphere through a 0.2 μm filter.

The lysate containing levansucrase is filtered though a sterile 0.2 μm filter and then added to the reactor. The enzyme converts sucrose into levans, generating glucose as a by-product. Some sucrose remains unreacted. The final levan mixture is removed from the reactor through FDA approved hollow fiber membranes.

### III. Resin Treatment

The levan solution from the hollow fiber/synthesis stage is rendered colorless and odorless using two FDA approved resins: Amberlite IRA958C1 and XAD16HP. The resin columns are set up in series. The levan solution has a low pH (3.8-4.2) after it comes through the IRA958C1. The pH of the levan solution is raised to 6.5-7.5 using NaOH. After the pH is adjusted, a UV-VIS scan is used to monitor reduction of impurities.

### IV. Nanofiltration

Separation of the simple sugars from the levans is achieved by using spiral wound nanofiltration membranes composed of FDA approved materials. The levan solution is concentrated, diafiltered against water, then concentrated again. The final levan pool is pumped through a 0.2 μm filter, and then spray-dried.

### V. Processing Aids and Process Chemicals

The processing aids used in the production of levans consist of 0.2 μm polypropylene filters, polysulfone hollow fiber membranes (AG Tech), polyethersulfone spiral wound nanofiltration-membranes (Osmonics/Desal), and Amberlite IRA958C1 and Amberlite XAD16HP resins (Rohm and Haas). Process chemicals include sodium hydroxide for pH adjustment, sodium hypochlorite for sanitization, and 2-propanol for sanitization.

### 5.2.2 EXAMPLE 2

### PRODUCTION OF BRANCHED FRUCTANS

### I. Process Description

Branched fructans are made in an enzymatic reaction that uses sucrose and fructooligosaccharides (FOS) as the starting materials. A mixture of branched fructans of varying saccharide units (DP 3 to 20 or more), glucose, fructose, difructose, and sucrose, is synthesized by the action of a levansucrase (from B. subtilis) upon sucrose and FOS. The recombinant enzyme is expressed in E.coli JM109.

The branched fructan mixture and the simple sugars (sucrose, glucose, and difructose) are separated from protein using hollow fiber (HF) membranes. Separation of the simple caloric low molecular weight sugars from the branched fructans is achieved by nanofiltration on spiral wound membranes. The branched fructans are rendered colorless and odorless by resin treatment. The clear, colorless branched fructan solution is then spray-dried to give a white, powdered product.

All materials used throughout the process are stainless steel, polypropylene, or high-density polyethylene. A UV-VIS scan is performed on each batch of branched fructans to monitor for the presence of impurities.

### II. Synthesis

The sucrose input is supplied as crude sugar, syrup, molasses, corn syrup or equivalent sucrose source, and is mixed with water, and then heated to approximately 30-35° C. to dissolve all of the sucrose. Inulin (FOS) is added to the mixture and dissolved. The solution is pumped into the reactor from a mix tank through a 0.2 μm polypropylene filter.

The reactor is a stainless steel closed tank with an insulating water jacket to allow for temperature control during operation. The pressure in the headspace is kept at equilibrium with the atmosphere through a sterile 0.2 μm filter.

The levansucrase enzyme is filtered though a sterile 0.2 μm filter and then added to the reactor. The enzyme converts sucrose into branched fructans, generating glucose as a by-product. Some sucrose remains unreacted. The final branched fructan mixture is removed from the reactor through FDA approved hollow fiber membranes.

### III. Resin Treatment

The branched fructan solution from the hollow fiber/synthesis stage is rendered colorless and odorless using two FDA approved resins: Amberlite IRA958C1 and XAD16HP. The resin columns are set up in series. The branched fructan solution has a low pH (3.8-4.2) after it comes through the IRA958C1. The pH of the branched fructan solution is raised to 6.5-7.5 using NaOH. At this step in the production, a UV-VIS scan is used to monitor for impurities.

### IV. Nanofiltration

Separation of the simple sugars from the branched fructans is achieved by using spiral wound nanofiltration membranes composed of FDA approved materials. The branched fructan solution is concentrated, diafiltered against water, then concentrated again.

The final branched fructan pool is pumped through a 0.2 μm polypropylene filter, and then spray-dried.

Following resin treatment, the branched fructan is analyzed, for example by any one or more of HPLC, MALDI-TOF or other mass spectroscopic technique, gel-permeation or affinity chromatography, IR spectroscopy, electrophoresis, NMR spectroscopy, dynamic light-scattering analysis, or other suitable analytical technique known to those of skill in the art.

### V. Processing Aids and Process Chemicals

The processing aids used in the production of branched fructans consist of 0.2 μm polypropylene filters, polysulfone hollow fiber membranes (AG Tech), polyethersulfone spiral wound nanofiltration membranes (Osmonics/Desal), and Amberlite IRA958C1 and Amberlite XAD16HP resins (Rohm and Haas). Process chemicals include sodium hydroxide for pH adjustment, sodium hypochlorite for sanitization, and 2-propanol for sanitization.

### EXAMPLE 3

### REPRESENTATIVE MALDI-TOF SPECTRA OF BRANCHED AND UNBRANCHED FRUCTANS OF THE PRESENT INVENTION

The synthetic methods described above produce fructans having a mode of distribution of DP. The fructans of the present invention therefore comprise, unless otherwise noted, a mode of distribution of DP. The distribution of the synthetically-produced fructans is characterized using time-of-flight mass spectrometry, as shown in FIGS. 12-14.

Major peaks indicate monovalent ions of fructan species associated with a single sodium ion (mass 22.99). Smaller peaks clustering close to, and mainly at lower m/z ratios, represent other adducts or salts. Each fructosyl unit within a chain contributes a mass of approximately 168. Hence, the distribution of degrees of polymerization of a preparation of fructan can be inferred, at least qualitatively, from inspection of the MALDI-TOF spectrum. In these figures, DP is indicated by the major peaks, for which the highest and lowest degrees of polymerization are identified by the large numerals.

FIG. 12 shows a preparation of long-chain levan, herein termed “4010LI40,” which is prepared as described above using sucrose at an initial concentration of 60% (w/v), and at a temperature of 50° C. From the MALDI-TOF spectrum it can be inferred that DP ranges from about 3 to about 29.

FIG. 13 shows a preparation of short-chain branched fructan, herein termed “4030LI40,” which is prepared as described above using sucrose at an initial concentration of 20% (w/v), in the presence of 40% inulin. From the MALDI-TOF spectrum it can be inferred that DP ranges from about 3 to about 14. This preparation is further characterized as described below using branch analysis and size-exclusion/light-scatter.

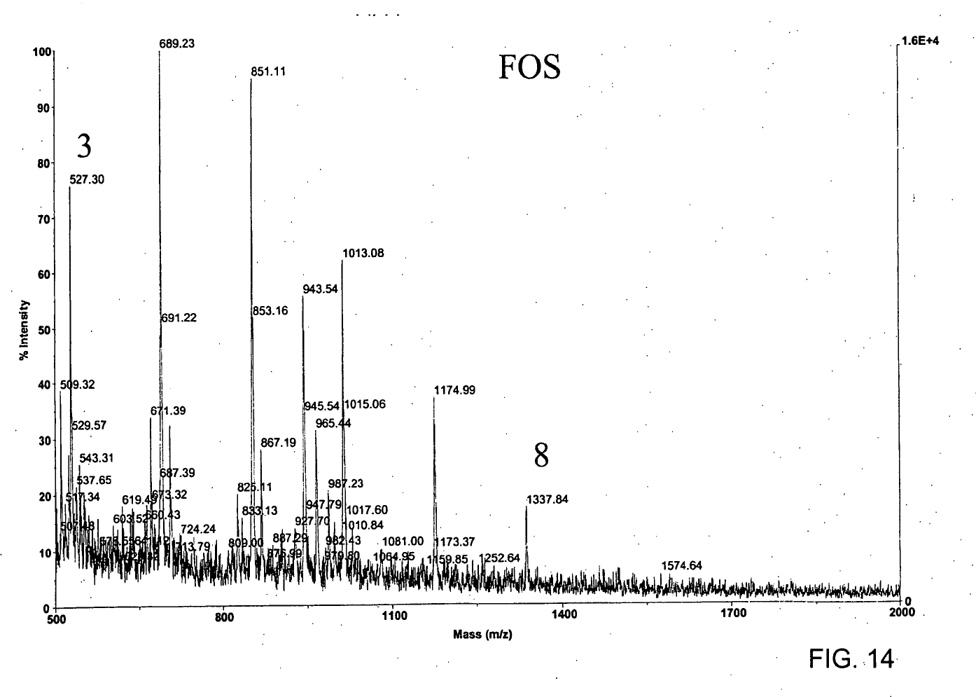

FIG. 14 shows a preparation of FOS, high FF FOS “meji,” which has a mode DP of approximately 4. From the MALDI-TOF spectrum it can be inferred that DP ranges from about 3 to about 8.

### EXAMPLE 4

### 5.2.4 SIZE-EXCLUSION/LIGHT-SCATTERING ANALYSIS OFBRANCHED AND UNBRANCHED FRUCTANS OF THE PRESENT INVENTION

The fructans of the present invention therefore comprise, unless otherwise noted, a distribution of DP. The distribution of the synthetically-produced fructans is characterized using size-exclusion/light-scattering analysis, which is more suited to quantitative analysis than is time-of-flight mass spectrometry.

FIG. 16 shows size-exclusion/light-scattering analysis applied to a preparation of short-branched fructan or branched FOS, herein termed “4030LI40.” In FIG. 16A, the mode DP can be inferred from the molar mass at which the cumulative weight fraction is increasing most rapidly, which is approximately at the midpoint. For this sample, a molar mass moment (Mw) of 1,067 is calculated, consistent with a mode DP of about 6. FIG. 16B shows raw data points fitted to a first-order model.

FIG. 15 shows size-exclusion/light-scattering analysis applied to a preparation of long-chain unbranched levan, herein termed “4020LI40.” In FIG. 15A, the mode DP can be inferred from the molar mass at which the cumulative weight fraction is increasing most rapidly, which is approximately at the midpoint. For this sample, a molar mass moment (Mw) of 2,523 is calculated, consistent with a mode DP of about 20. FIG. 15B shows raw data points fitted to a first-order model.

### EXAMPLE 5

### 5.2.6 LINKAGE ANALYSIS OF BRANCHED AND UNBRANCHED FRUCTANS OF THE PRESENT INVENTION

Chemical linkage analysis is applied to two preparations of levan (“4010LI40,” which is synthesized using 60% (w/v) sucrose at 50° C., and “4020LI40,” which is synthesized using 60% (w/v) sucrose at 30° C.) and two preparations of branched fructan (“5028BI50,” which is synthesized using a 1:1 ratio of inulin to sucrose, and “4030LI50,” which is synthesized using a 1:0.5 ratio of inulin to sucrose).

The results from linkage analysis are presented in Table 2 as the normalized mole % for each of the residues found.

TABLE 2

| | | | 5028BI50 | 4030LI40 |
|---|---|---|---|---|
| | | | (Long- | (Short- |
| | 4010LI40 | 4020LI40 | Branched | Branched |
| Residue | (Levan) | (Levan) | fructan) | fructan) |
| Red 6-Fru | 2.3 | 1.6 | 2.2 | 1.8 |
| Red 1-Fru | 0.4 | 0.4 | 1.2 | 2.0 |
| T-Fru | 5.4 | 6.6 | 13.7 | 13.0 |
| T-Glc | 5.7 | 4.6 | 18.5 | 15.2 |
| Red 1,6-Fru | 0.0 | 0.0 | 0.4 | 0.9 |
| 6-Fru | 74.4 | 77.5 | 41.3 | 27.7 |
| 1-Fru | 3.3 | 1.9 | 14.9 | 22.8 |
| 6-Glc | 0.7 | 2.0 | 1.7 | 2.9 |
| 1,6-Fru | 8.0 | 5.4 | 6.0 | 13.7 |

Abbreviations: Red, reducing terminal; T, non-reducing terminal; Fru, fructose; and Glc, glucose.

The largely unbranched nature of the levan preparations is apparent from the high proportion of 6-Fru, and low proportions of 1-Fru and non-reducing terminals. The ratio of 1-linked fructose to 6-linked fructose therefore provides a convenient measure of branching. These results are shown in Table 3.

TABLE 3

| | | | | | 4030LI40 |
|---|---|---|---|---|---|
| | | | | 5028BI50 | (short- |
| Fructose | | 4010LI40 | 4020LI40 | (long-Branched | Branched |
| residue | Inulin | (Levan) | (Levan) | fructan) | fructan) |
| 1-linked | 60 | 4 | 2 | 18 | 25 |
| 6-linked | 0 | 77 | 94 | 44 | 30 |
| Ratio of 1- | — | 0.05 | 0.02 | 0.4 | 0.9 |
| to 6-linked | | | | | |

Using Table 3, the relative amount of branching can be inferred.

### 5.3 FAVORABLE EFFECT OF INVENTIVE FRUCTANS ON BIFIDOGENIC GROWTH

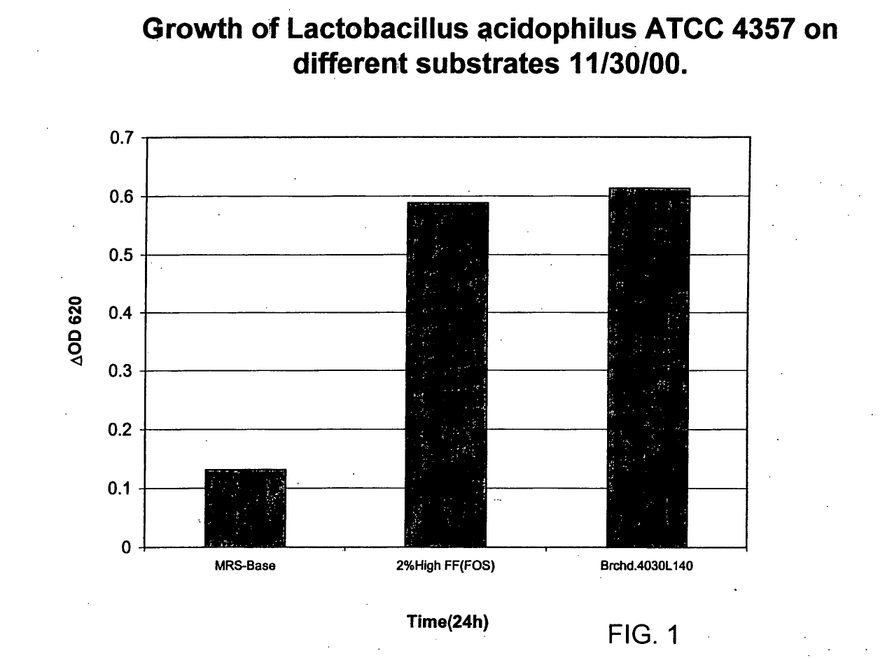

FIGS. 1-6 are non-limiting examples demonstrating that fructan compositions according to the present invention promote the growth of bifidogenic bacteria in vitro, including certain non-pathogenic Lactobacillus strains. In the examples which follow, the notation “FOS” denotes a mixture of inulins ranging from DP3 to DP9 and being predominately FFn. The notation DPn/FOS indicates a purified FOS. For example, the notation DP4/FOS denotes an FOS having DP4, or more typically, a distribution of fructans having a mode corresponding to DP4. In the present example, DP4/FOS is purified from a FOS mixture by size-exclusion chromatography.

### 5.3.1 EXAMPLE 3

Referring now to FIG. 1, the beneficial effect of fructans according to the invention are evaluated by comparing the proliferation of Lactobacillus acidophilus ATCC 4357 resulting from metabolism of fructan compositions in accordance with the invention as the energy source. The proliferation of the bacteria is evaluated without a sugar source. The bacteria are allowed to grow in liquid culture comprising MRS (deMan, Rogosa, and Sharpe) media and 12% CO₂ at 37° C. Proliferation is monitored by light scattering at 620 nm and recorded as an optical density. The formula for MRS broth, (minus dextrose “base media,” per liter) is: 10 g BactoProteose Peptone No.3; 10 g Bacto beef extract; 5 g Bacto yeast extract; 1 g Sorbitan monooleate complex; 2 g ammonium citrate; 5 g sodium acetate; 0.1 g magnesium sulfate; 0.05 g maganese sulfate; and 2 g potassium phosphate, dibasic. The final pH is 6.5 at 25° C.

More specifically, in this experiment, a short-chain branched fructan having DP of about 6, a FOS mixture, a DP 4/FOS, a long-chain branched levan having DP of about 15 (5028BI50), and a first and second unbranched levan having DP20 (4010LI40 and 4020LI40)are. The results of linkage analysis of these preparations is described in section 5.2.6. The levans and the branched fructans were synthetically made in accordance with the examples above. The first and second levan having DP20 are distinguishable in that the first levan was produced at a temperature of 50° C., whereas the second levan was produced at 30° C. The FOS is a Raftilose™ product from chicory. Accordingly the FOS is a mixture of fructans and is predominately Fn rather than GFn. The DP4/FOS was purified from FOS by size-exclusion chromatography. The levans and branched fructans were enzymatically produced, they all represent a distribution of fructans, with mode corresponding to the indicated DP (FOS is a mixture of fructans, each having a distribution and mode corresponding to a DP of from 3-9), and are all predominately Fn.

The results reported in FIG. 1 show that short-chain branched fructans were more effective in promoting the growth of the bacteria than 2% high FF/FOS.

### 5.3.2 EXAMPLE 4

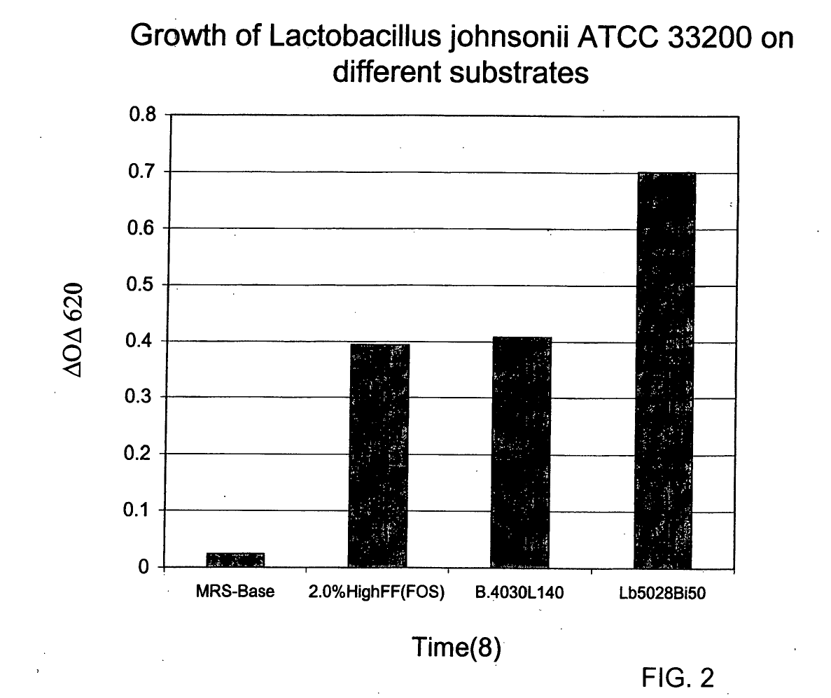

Referring now to FIG. 2, the beneficial effect of fructans according to the invention is evaluated by comparing the proliferation of Lactobacillus johnsonii ATCC 33200 resulting from metabolism of fructan compositions in accordance with the invention as the energy source, with the proliferation of the bacteria without a sugar source. The bacteria are allowed to grow in liquid culture comprising MRS media and 5% CO₂ at 37° C. Proliferation is monitored by light scattering at 620 nm and recorded as an optical density.

In this experiment, the same long-chain branched levan ((5028BI50), as well as the same FOS of Example 1 were used.

According to the results reported in FIG. 2, the fructan compositions of the invention were each beneficial to the proliferation of this bacteria, both branched levans were more beneficial for the proliferation of this bacteria.

### 5.3.3 EXAMPLE 5

### 5.3.4 EXAMPLES 6 AND 7

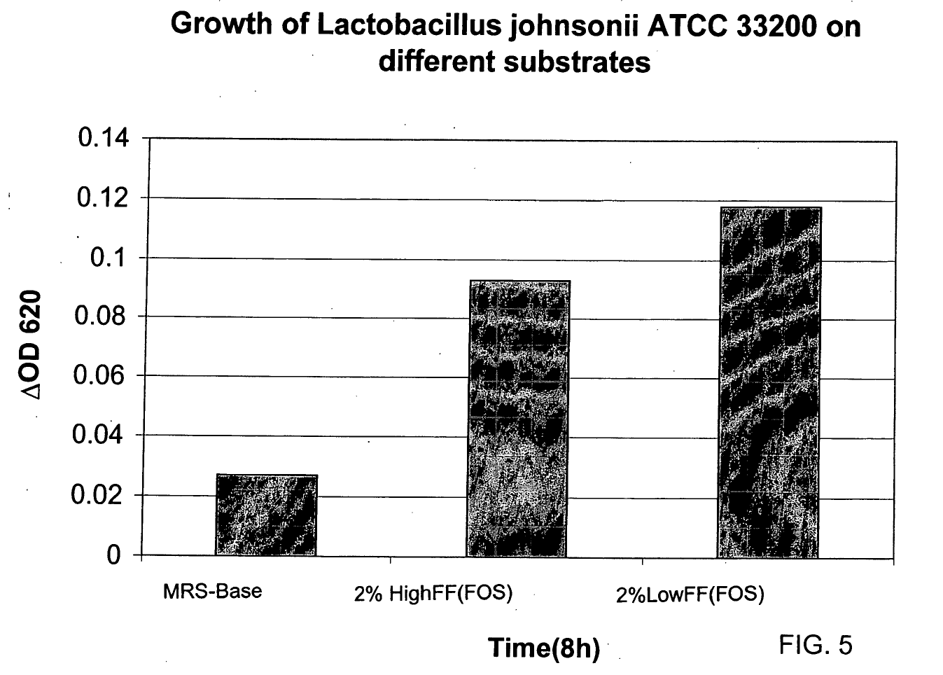

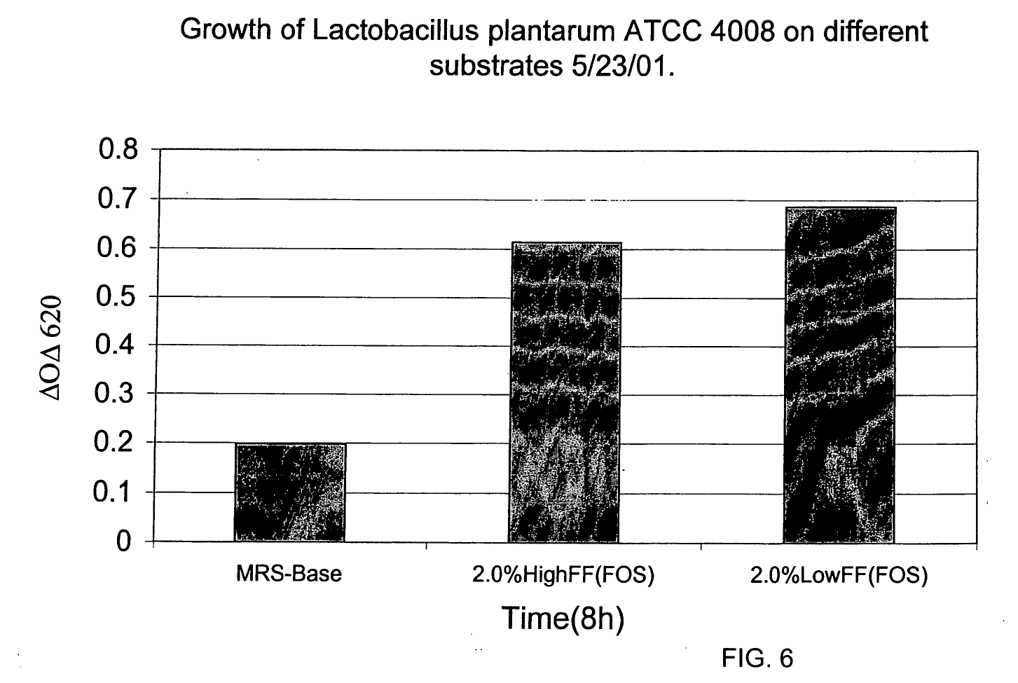

Referring now to FIGS. 5 and 6, the effect on the growth Lactobacillus johnsonii ATCC 33200 (FIG. 5) and Lactobacillus plantarum ATCC 4008, of two FOS preparations is tested compared to MRS-base. As before, the bacteria were allowed to grow in liquid culture comprising MRS media and 5% CO₂ at 37° C. Proliferation was monitored by light scattering at 620 nm and recorded as an optical density. The “High FF” FOS is a Raftilose™ preparation that is substantially Fn, and the “Low FF” FOS is an enzymatic preparation containing substantial amounts of GFn.

The results reported in FIGS. 5 and 6 show an enhancement of growth using FOS containing GFn compared to predominantly Fn.

### 5.3.5 EXAMPLES 8 TO 13

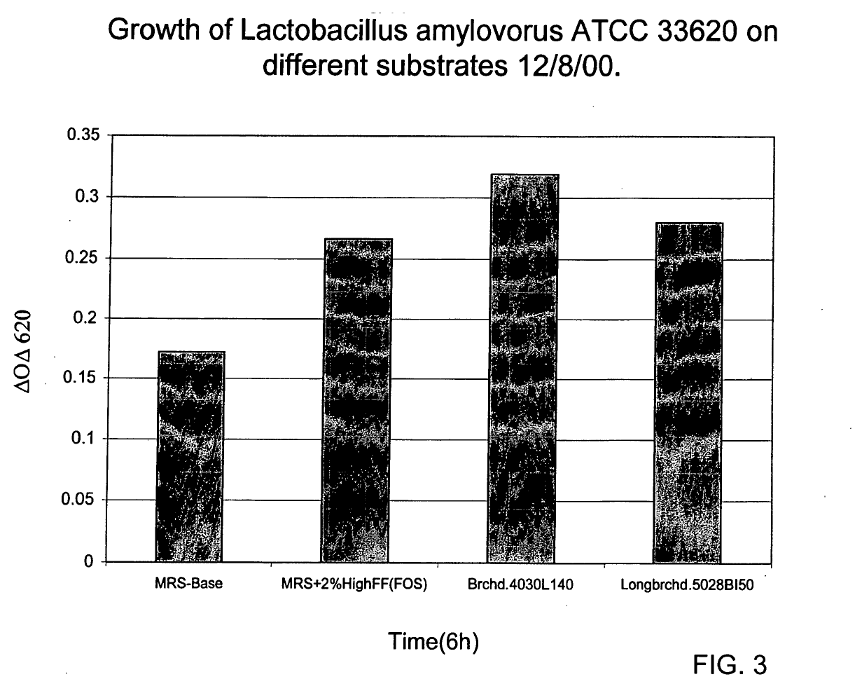

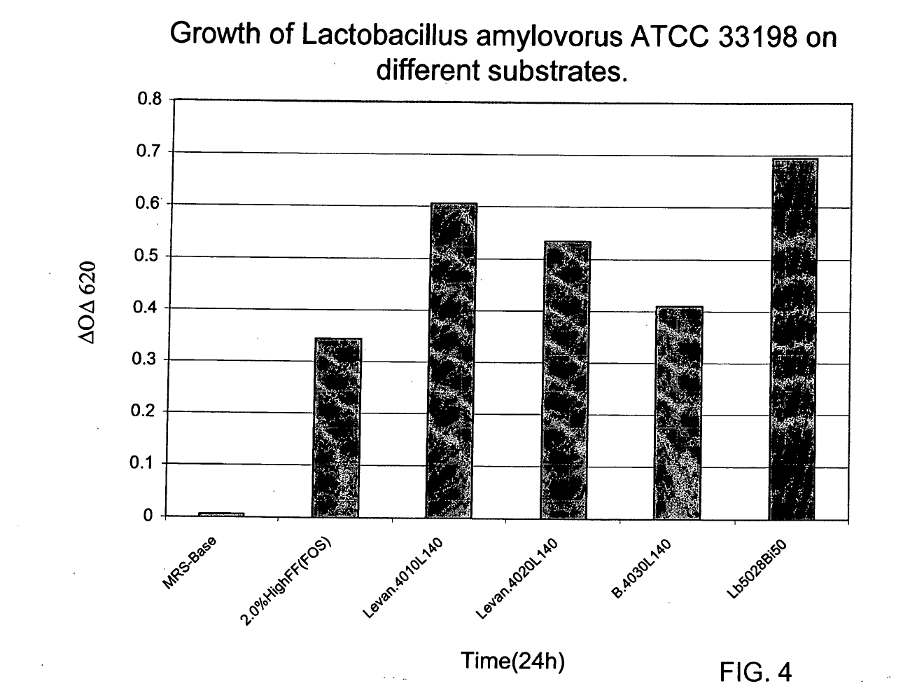

Referring now to FIGS. 3 and 4, the effects of fructans of the present invention on the growth of: Lactobacillus amylovorus ATCC 33198 (FIG. 4); Lactobacillus amylovorus ATCC 33620 (FIG. 3). As before, the bacteria were allowed to grow in liquid culture comprising MRS media and 5% CO₂ at 37° C. Proliferation was monitored by light scattering at 620 nm and recorded as an optical density.

Comparison with growth supported by glucose and sucrose as carbon sources shows that FOS and unbranched and brached levans are excellent carbon source for growth of these Lactobacilli in each case.

### 5.3.6 EXAMPLES 18 TO 22

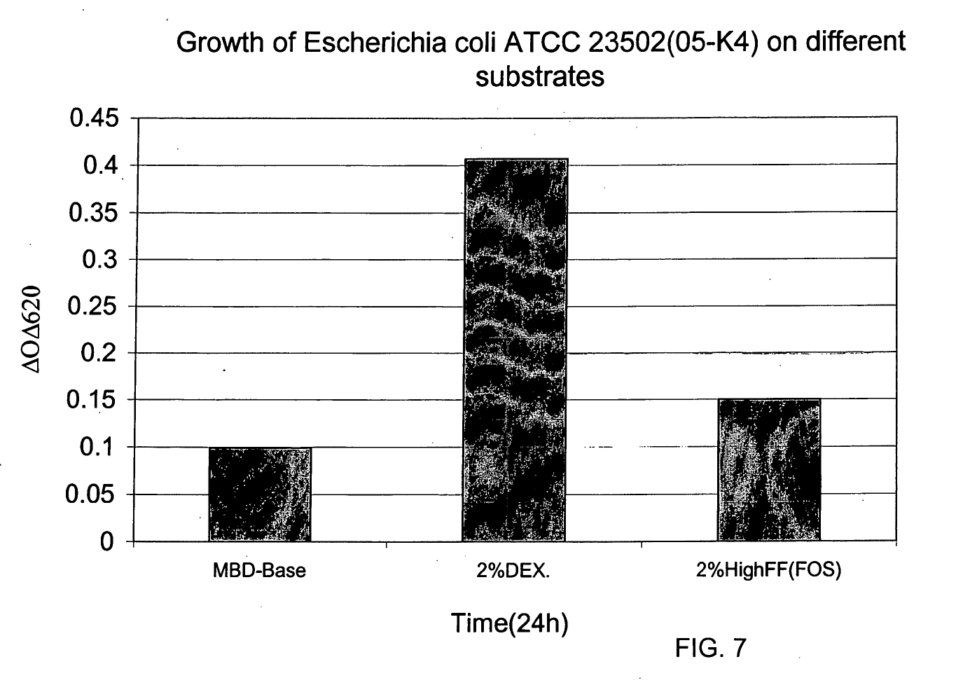

Referring now to FIGS. 7 to 9, the effect on the growth pathogenic bacteria was evaluated for the fructans described above in connection with Examples 1-6, and for 2% dextrose and 2% sucrose.

Escherichia coli is a leading cause of food poisoning and other diseases. Thus: enterotoxic E.coli (ETEC) causes gastroenteritis, or traveler's diarrhea; enteropathogenic E.coli (EPEC) causes infantile diarrhea; enterohemorrhagic E.coli (EHEC), e.g. EC O157:H7, causes hemorrhagic colitis; and enteroinvasive E.coli (EIEC), e.g. ATCC 23502(O5-K4) and U1-41(O5-K4), cause bacilliary dysentry. In these examples, fructans are shown to only poorly support the growth of certain strains of pathogenic E.coli compared to glucose or fructose as a carbon source.

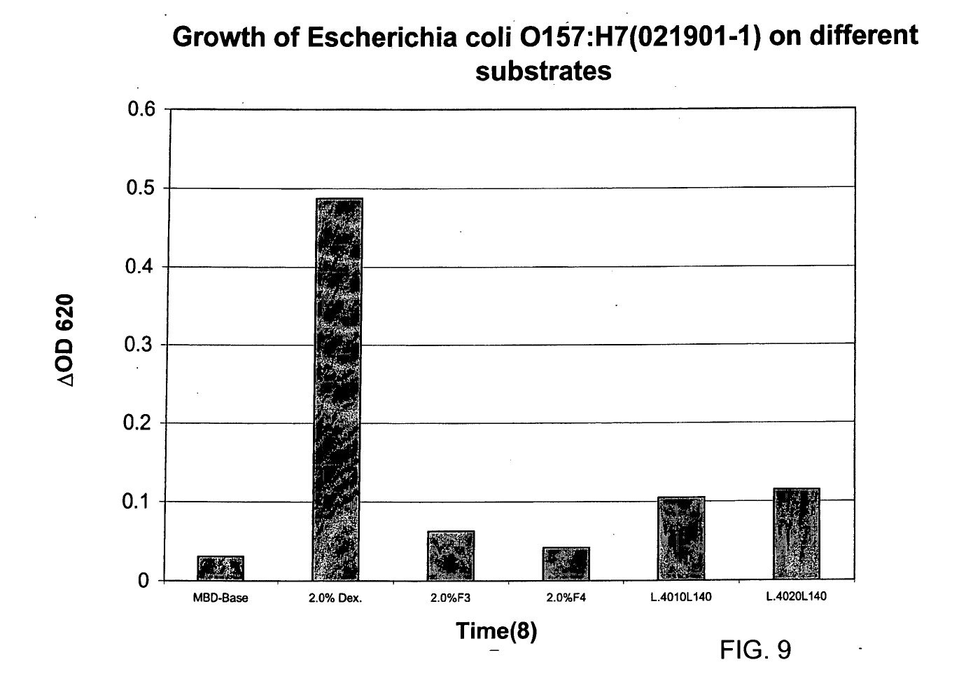

FIG. 9 illustrates that growth of E.coli O157:H7 is poorly supported by levans, and purified F-series FOS, compared to growth on dextrose.

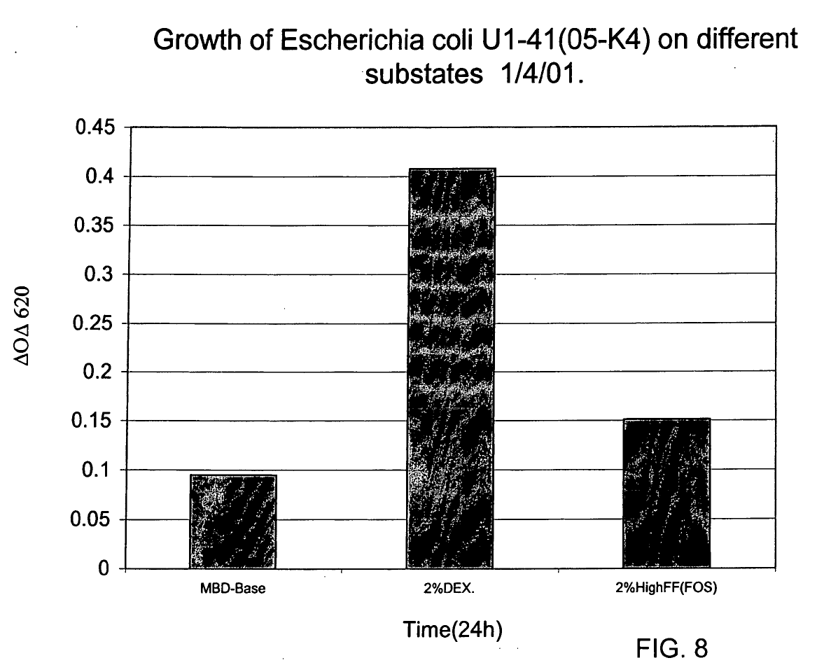

FIGS. 7 and 8 further illustrate that growth of E.coli ATCC 23502 (O5-K4) and U1-41(O5-K4) are both poorly supported by 2.0% FOS compared to 2.0% dextrose.

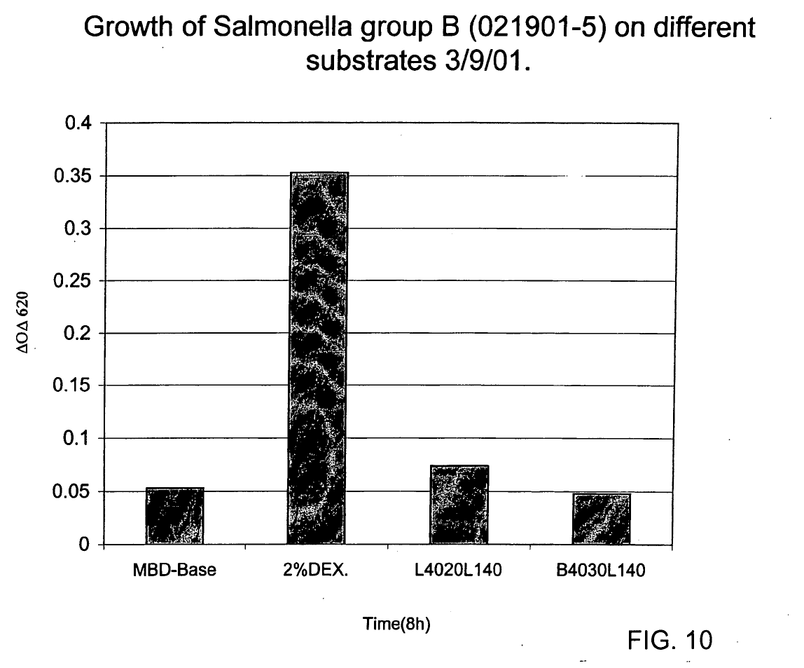

FIG. 10 illustrates that growth of Salmonella species group B(0211901-5), which causes food poisoning, is also poorly supported by the fructans of the present invention compared to growth on 2.0% dextran.

Therefore, it is shown that compounds of the present invention do not efficiently support the growth of pathogenic strains of E.coli or salmonella.

Branched FOS, FOS that are substantially GFn, and levans are a poorer carbon source for supporting the growth of pathogenic strains of E.coli or salmonella than FOS from natural sources or unbranched FOS. None of the compounds support growth as well as dextrose.

### 5.3.8 EXAMPLE 23

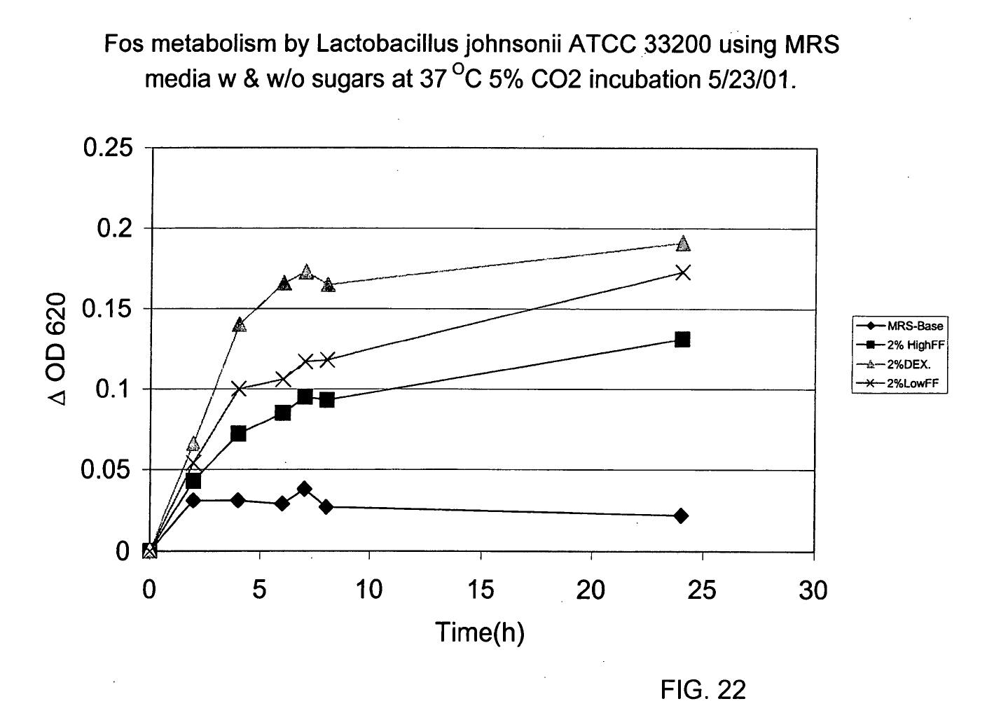

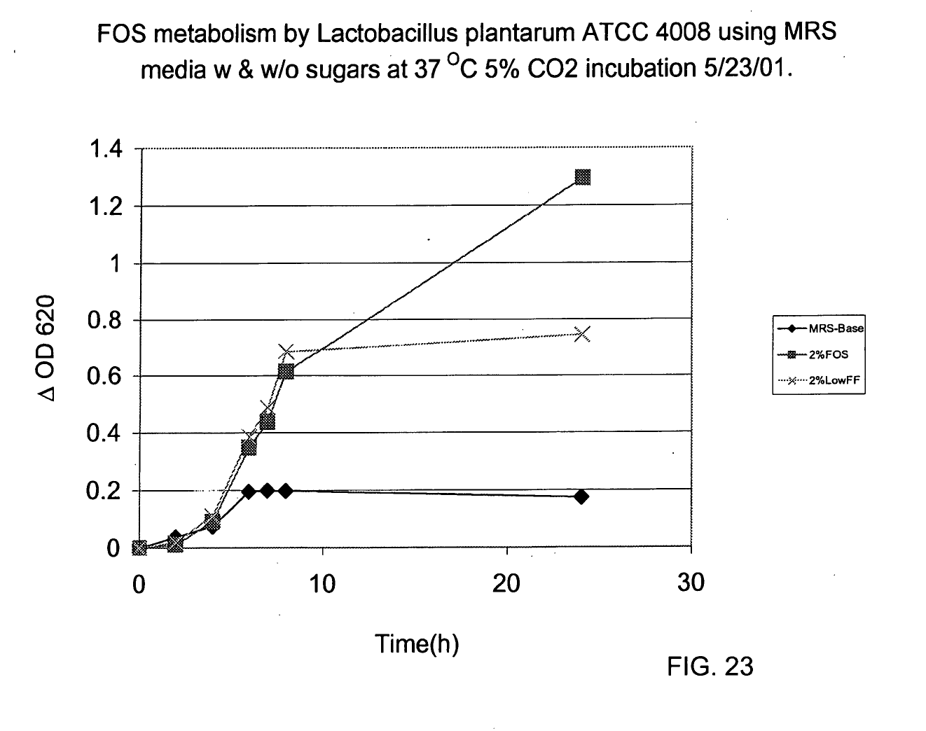

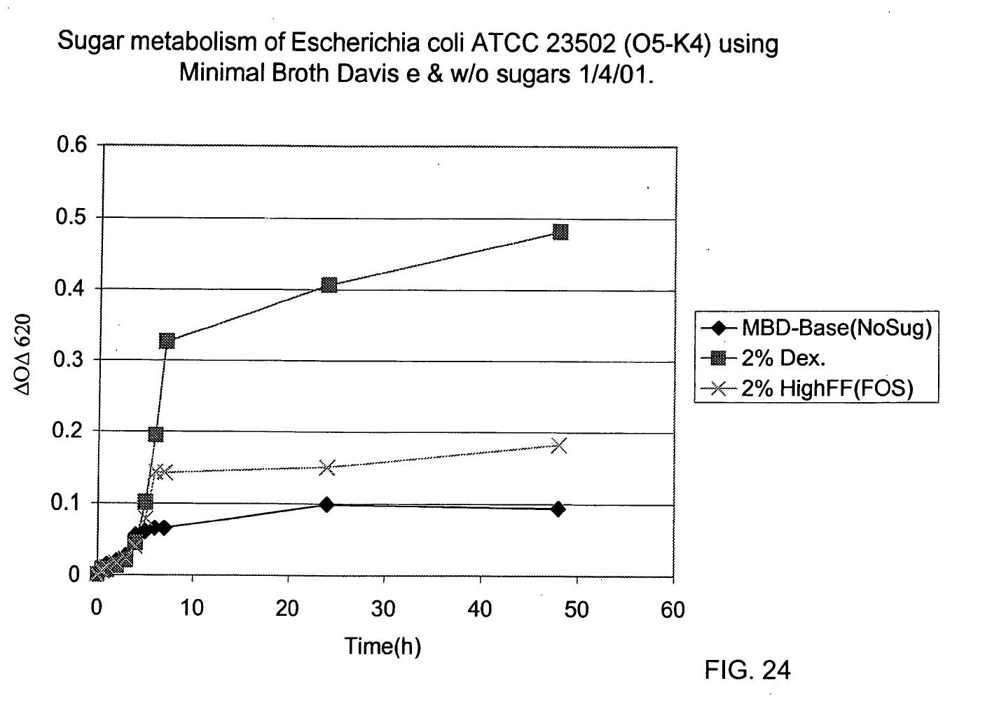

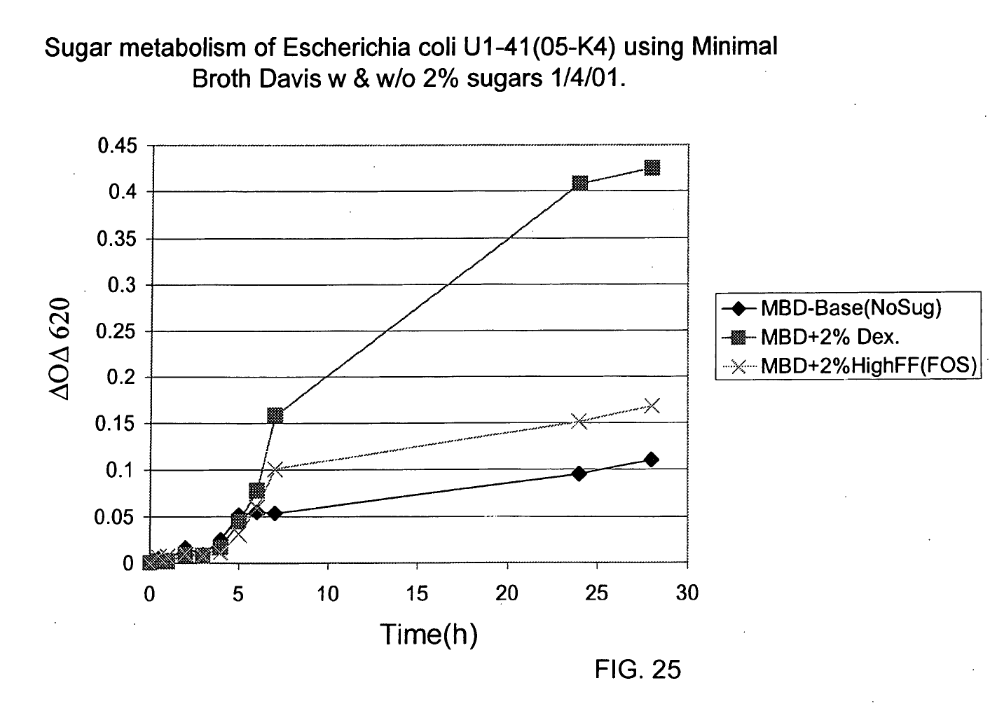

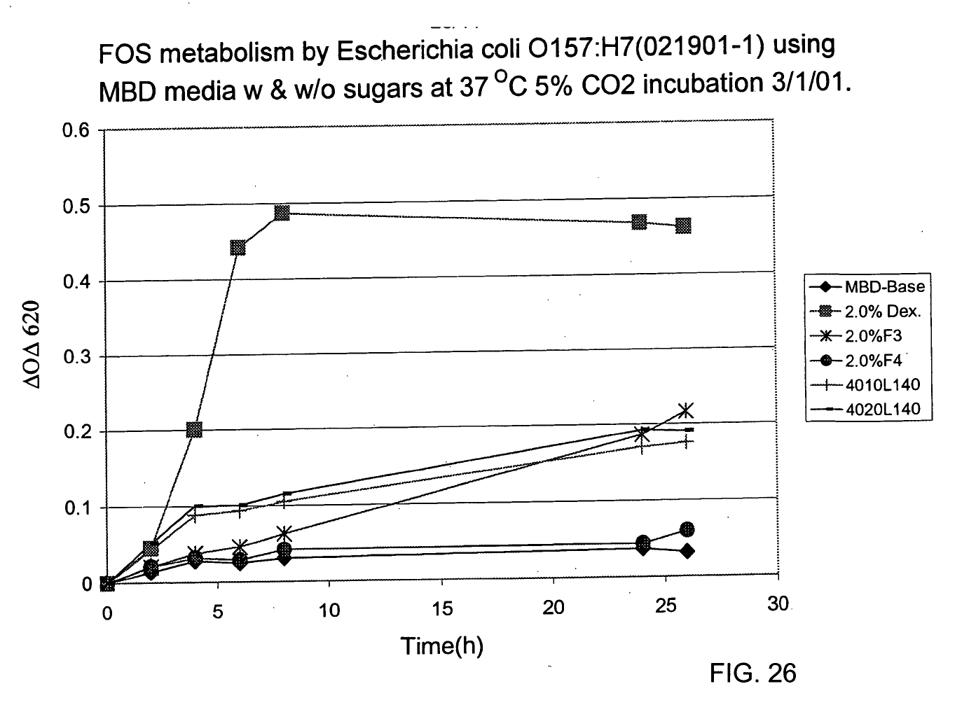

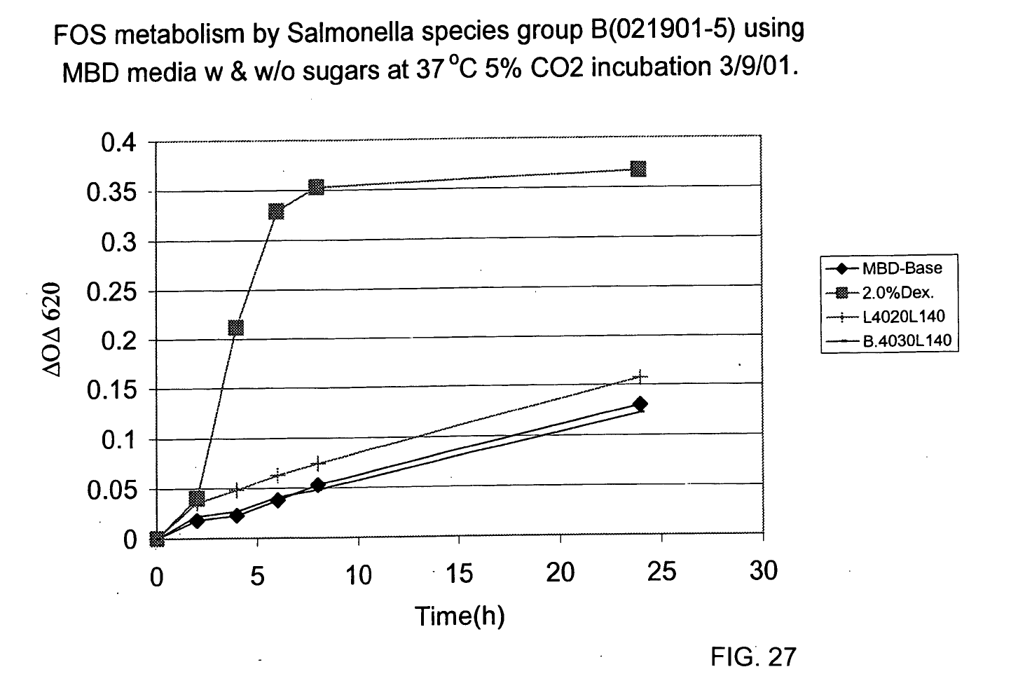

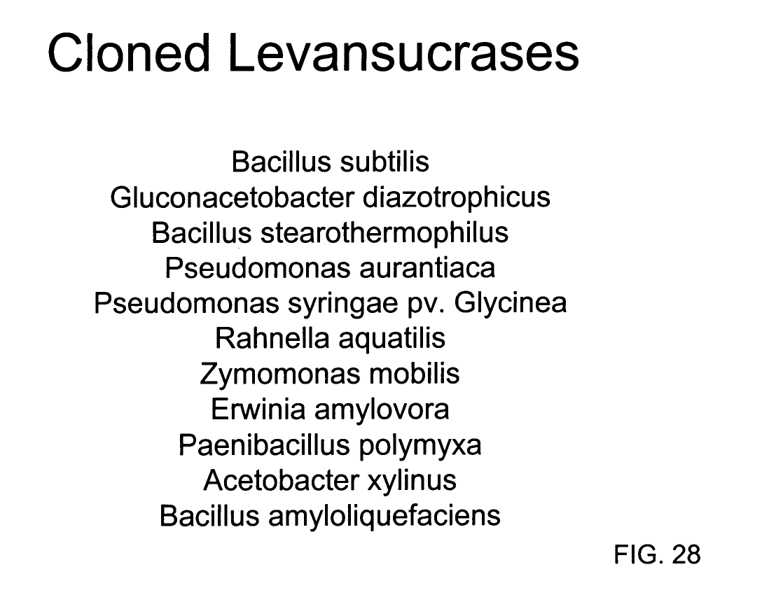

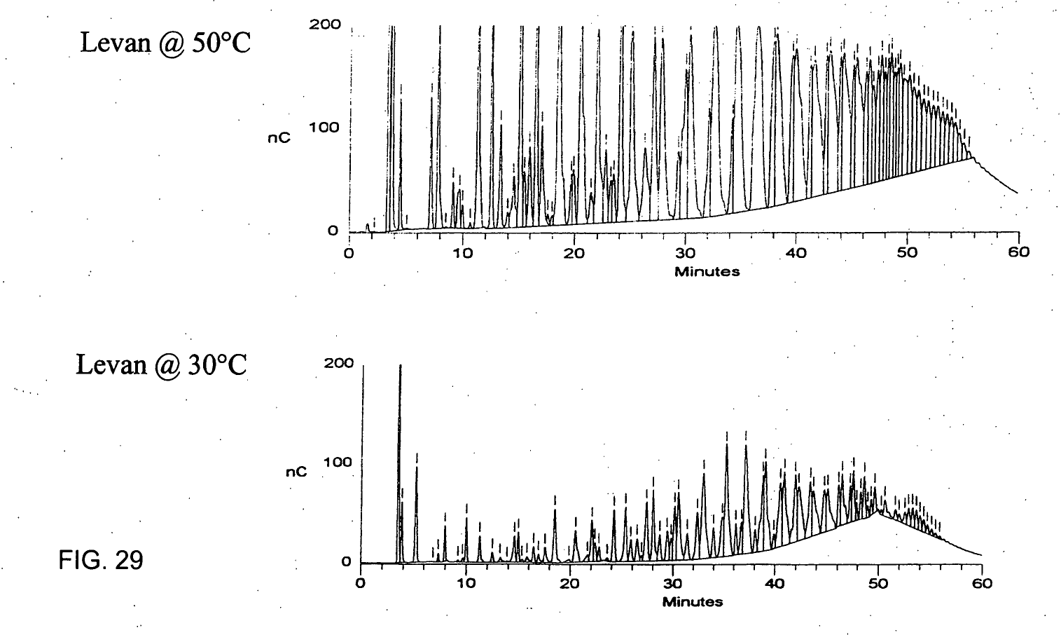

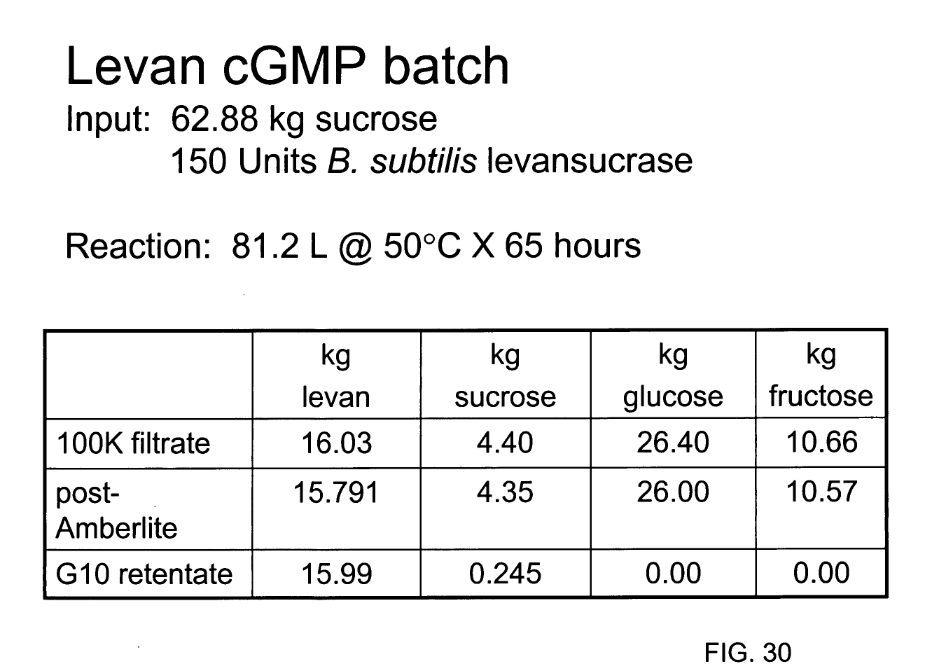

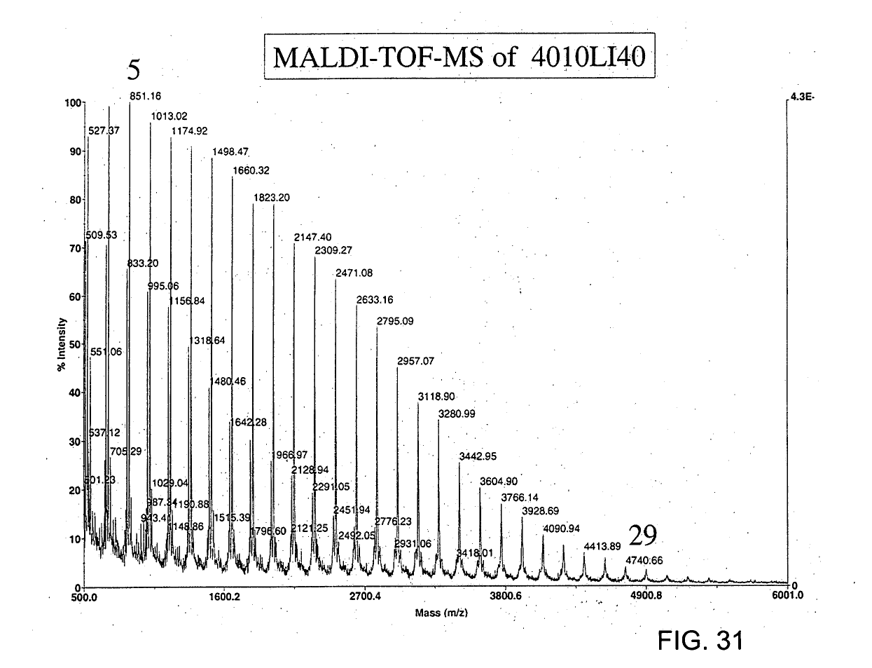

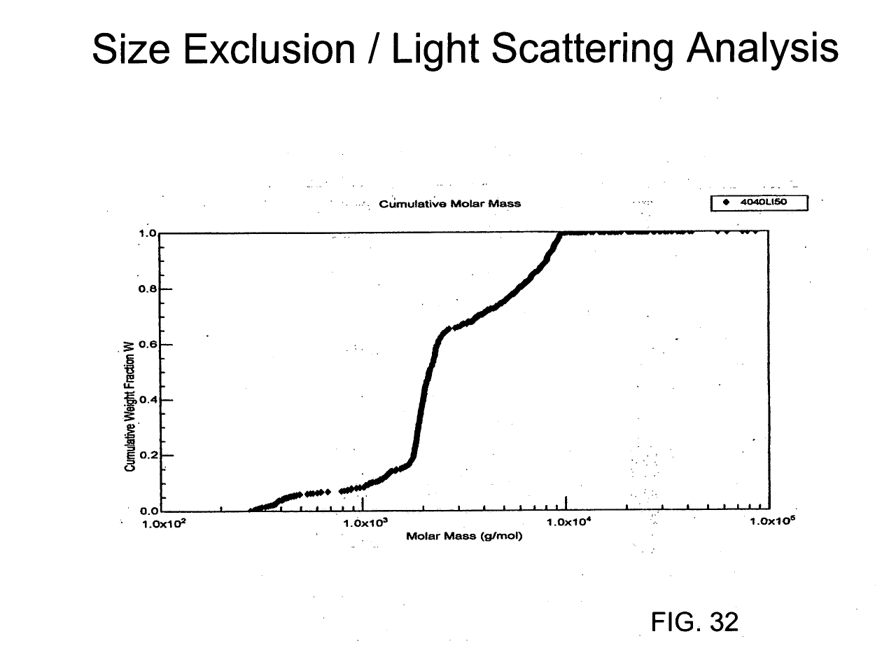

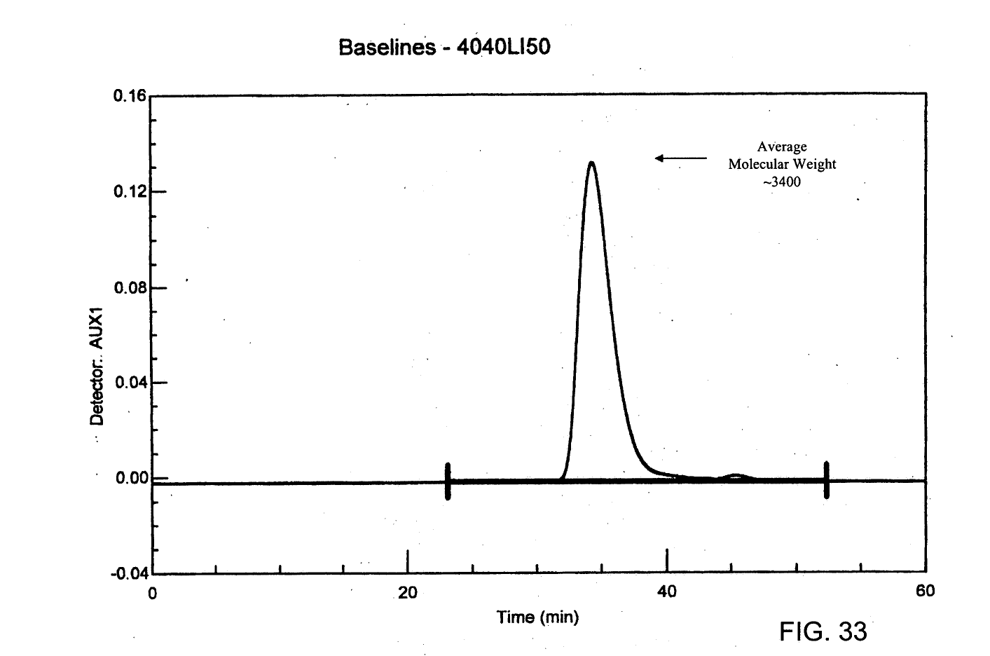

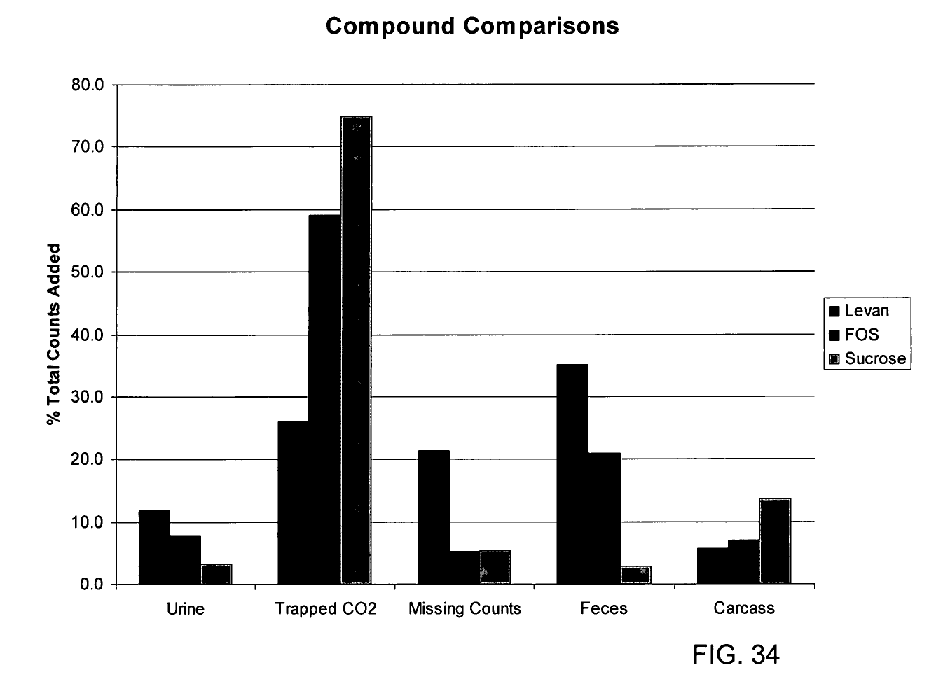

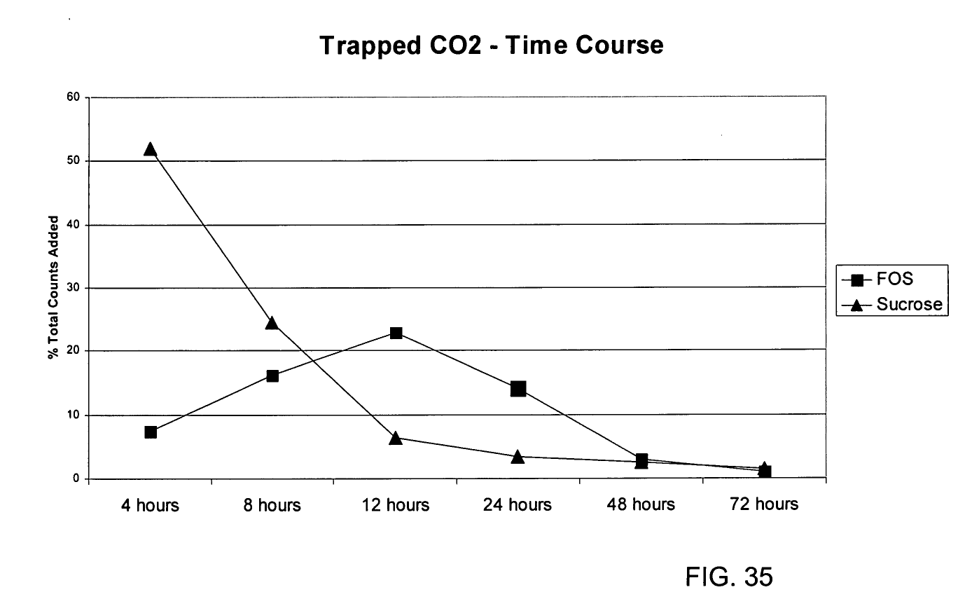

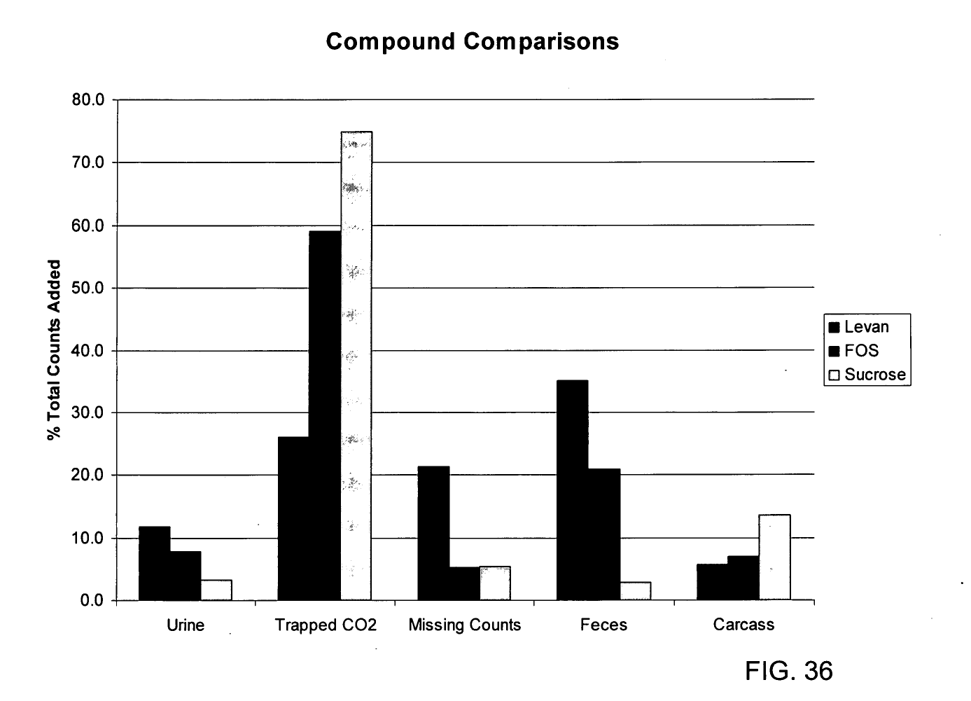

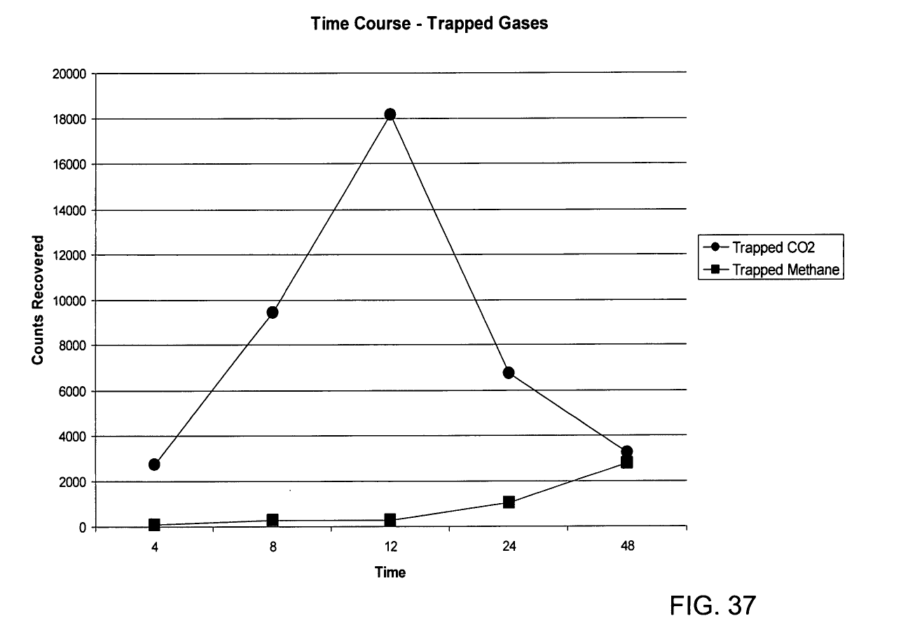

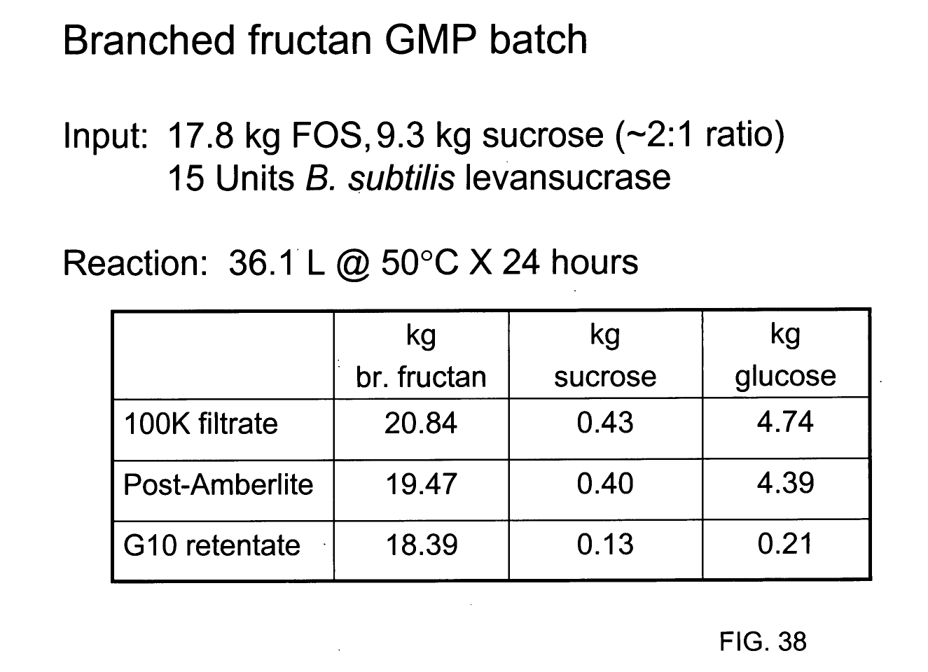

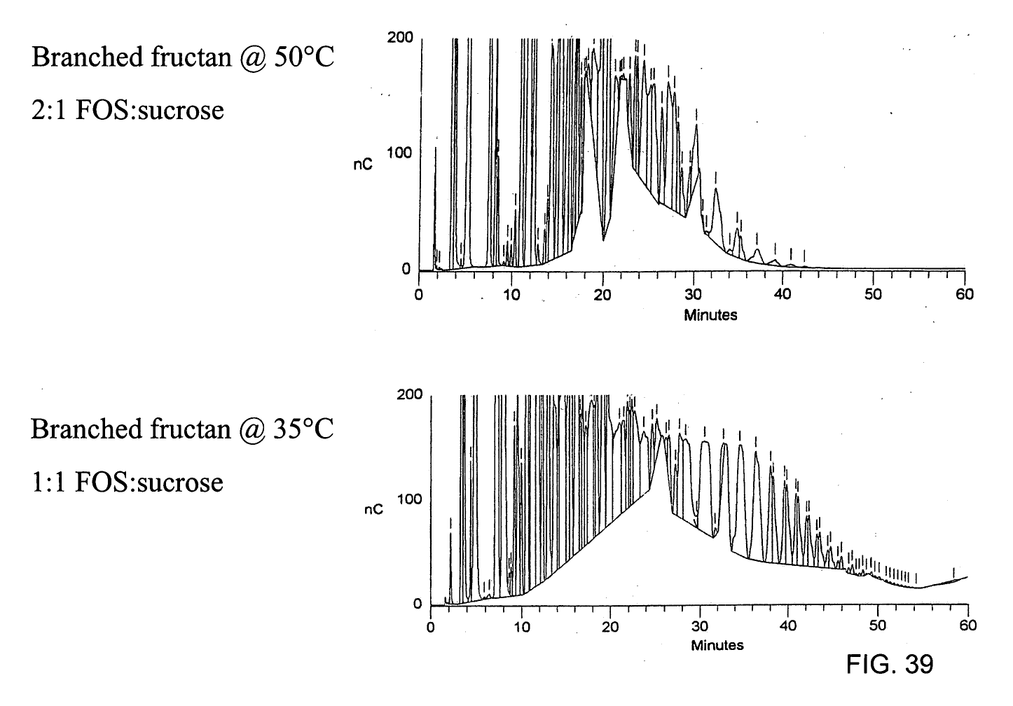

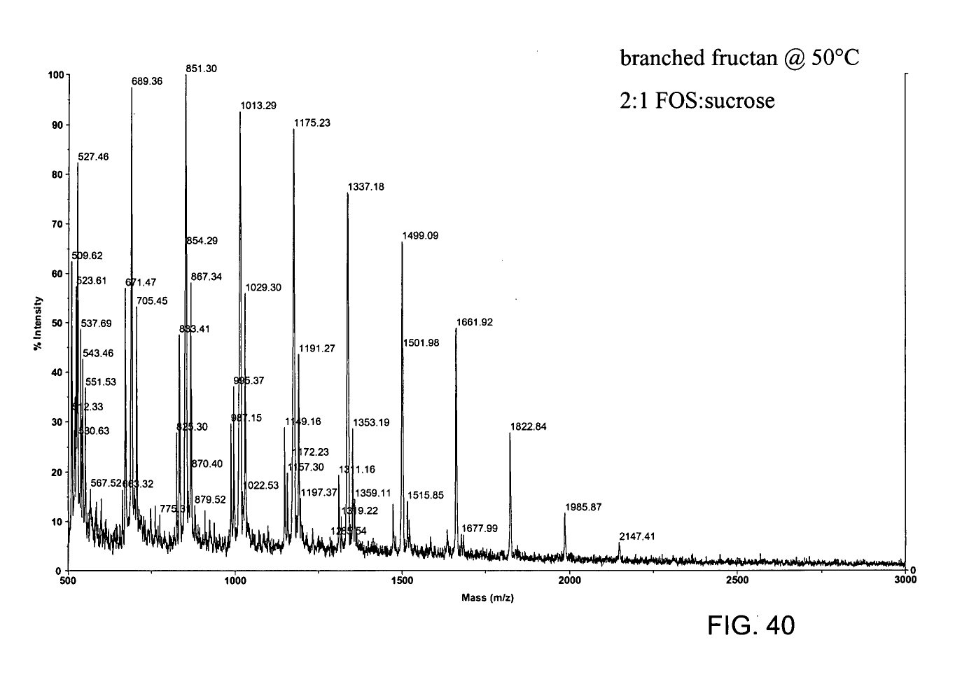

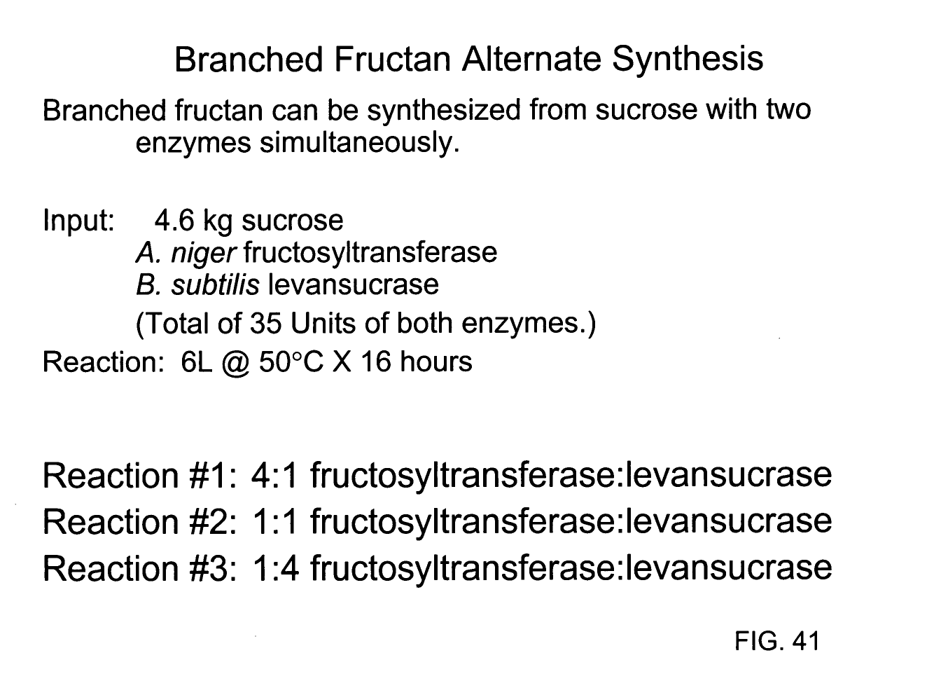

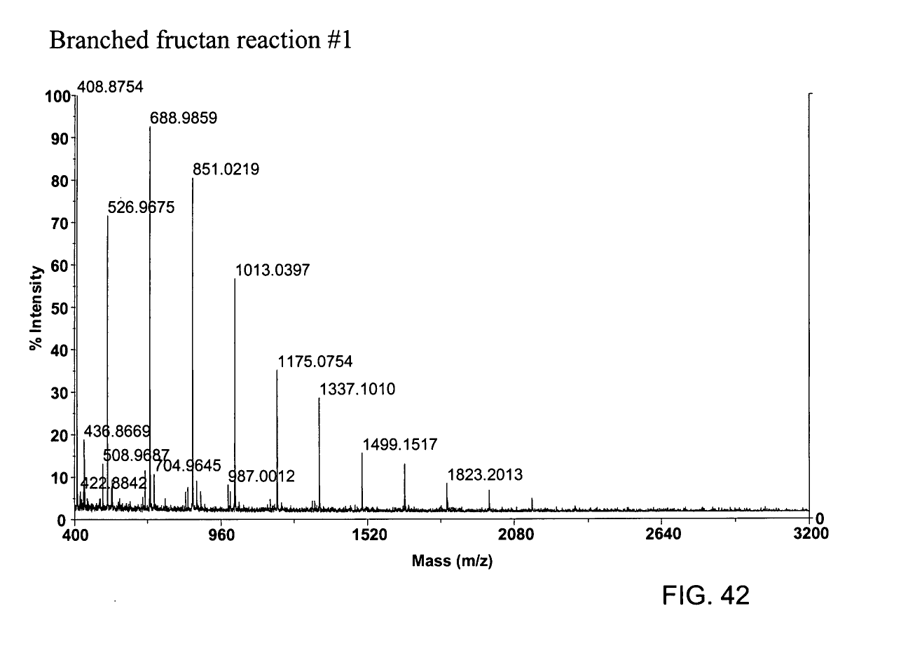

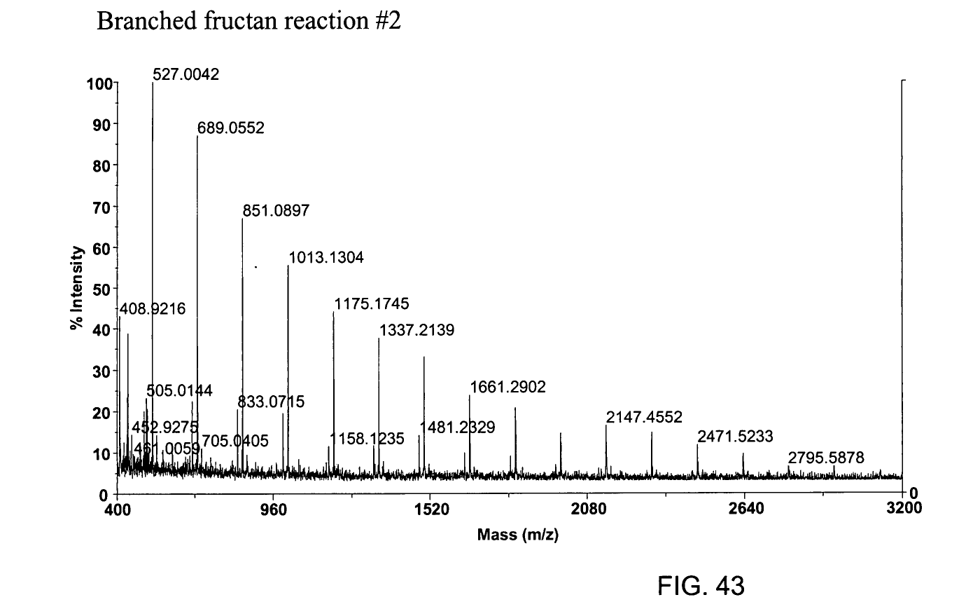

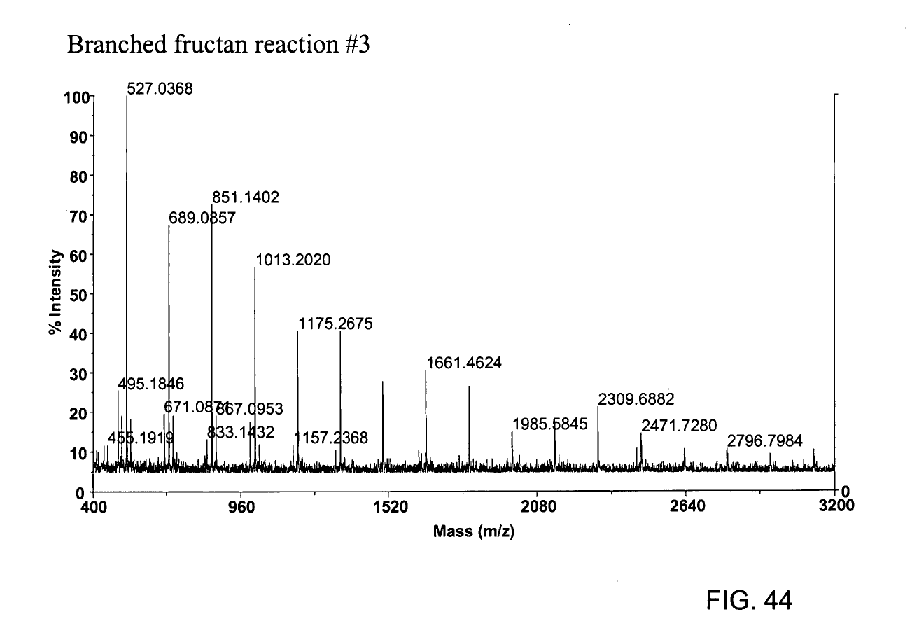

FIG. 22 illustrates the results of rat studies, in which rats were fed [¹⁴C]-radio-labeled FOS, levan, and sucrose. An exemplary preparation of branched FOS used in the above studies (5028BI50) is analyzed the previous examples, has a mode DP of 18, and a distribution of DP from about 3 to about 14.

The metabolic end-products of the feedings were monitored by sampling and collecting urine, expired air, feces, and carcass. In addition, a rinsing of the cage was performed to account for bodily fluids, uneaten food, and other potential losses of counts. The difference between the sum of these counts and the amount of counts administered was assumed to be accounted for as expired methane resulting from the metabolism of fructan by intestinal methanogenic flora.

The preferential metabolism of sucrose by the rat is shown by the high proportion of counts in expired air (CO₂) and in the feces and carcass. In contrast, approximately one-third of the fructan counts appear in the feces, and approximately a quarter in assumed methane. A much lower proportion of counts appear in the carcass, consistent with poor absorbtion and anabolic utilization of fructans by the rat. In this respect, branched FOS and levans are poorly metabolized by the rat. In short, the studies illustrate that branched FOS are metabolized less than half as efficiently as unbranched FOS and that branched FOS, levans, and glucans are generally less efficiently metabolized than sucrose and unbranched FOS.

The reported results should equally apply to humans. Although the experiments above were done in rats, sugar tolerance tests confirm that FOS are neither significantly digested nor significantly absorbed by humans; the estimated caloric value of FOS and inulin is less than 2 kcal per gram, and could range down to zero.

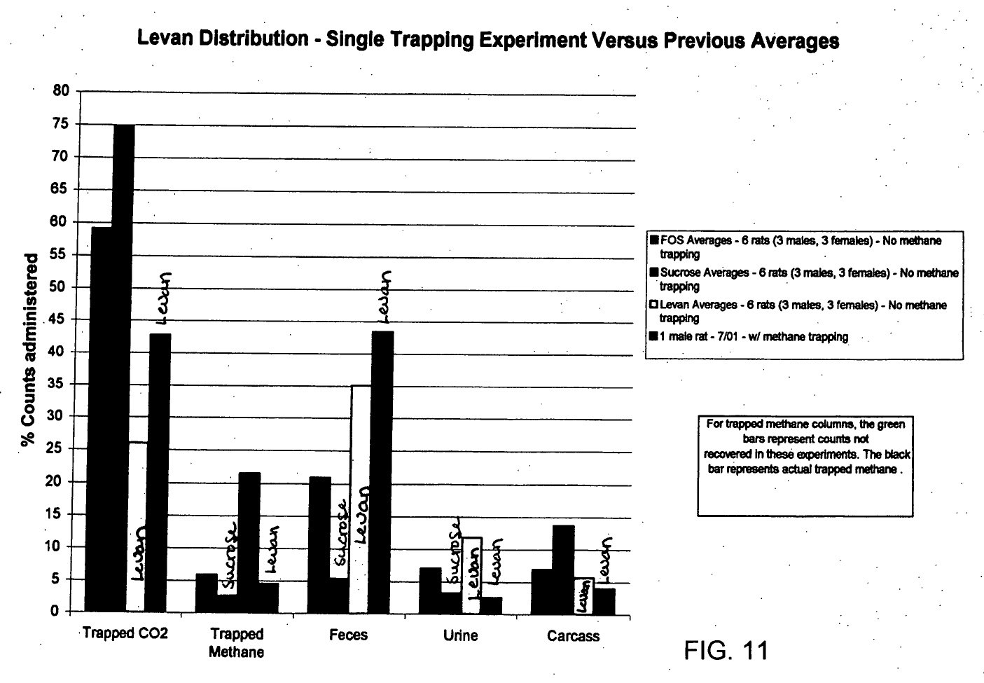

For some embodiments of the invention, it is an advantage that the fructans of the present invention are poorly metabolized by humans and animals. Thus, in some embodiments, the present invention provides low-calorie compositions, for example food additives land sweetening agents of low calorific value, which contribute less to the problem of obesity than sucrose (e.g. corn syrup). In some embodiments, the present invention provides reduced-gas compositions. In FIG. 11 it is shown that levans and branched FOS both produce more methane than FOS (unbranched) when used in rat feed.

As the examples illustrate, the compositions of the present invention can promote healthy microflora in humans and animals, in locations such as the intestine where bifidobacteria and lactic acid producing bacteria can be found (FIGS. 1-6), and can also disfavor the growth of pathogenic bacteria (FIGS. 7-10). Further, certain compositions according to the present invention produce less constipation than compositions having unbranched FOS. In certain embodiments, the compositions comprise branched fructans, which can be branched levans. Branched levans appear to be more poorly digested than unbranched fructans, thereby producing less gas or constipation (Gibson, G. R., Beatty, E. R., Wang, X. & Cummings, J. H. “Selective Stimulation of Bifidobacteria in the Human Colon by Oligofructose and Inulin” Gastroenterology (1995)108:975-82).

The compositions of the present invention can also be useful for promoting healthy microflora in other human internal cavities that are colonized by bacteria whose growth is promoted by fructans. For example, the compositions of the present invention can be useful in promoting the healthy microflora of the human vagina. In Example 1, FIG. 1, it is shown that the growth of Lactobacillus acidophilus, which is the most frequently isolated species of lactobacillus from the human vagina (see, McGroarty, J. A. “Probiotic use of lactobacilli in the human female urogenital tract”, FEMS Immunology and Medical Microbiology 6 (1993) 251-264, and references therein), is promoted by fructans of the present invention. Thus L.acidophilus is a major component of the normal human vaginal flora and this flora has been shown to be important in prevention of urinary tract infections, bacterial vaginosis, post antibiotic infections, infections associated with the use of spermicidal preparations, and in the general maintenance of the correct vaginal pH. See, McGroarty, J. A. “Probiotic use of lactobacilli in the human female urogenital tract”, FEMS Immunology and Medical Microbiology 6 (1993) 251-264, and references therein. Accordingly, although the fructan compositions of the invention are primarily intended for oral consumption, they can be incorporated into creams, for example, for vaginal application to promote healthy microflora.

The compositions of the present invention can be ingested or applied through the use of troches, cremes, suppositories, and other means known to those of skill in the art. For the maintenance of healthy microflora, regular and repeated ingestion is preferable to single application.

In certain embodiments, a sweetener is provided that contains a trichlorinated fructose, such as for example Sucrolose. Sucrolose is 600× sweeter than sucrose, and is therefore conveniently combined with non-chlorinated fructans that are other embodiments of the present invention and which are much less sweet, in order to provide, for example, a sweetener of comparable sweetness to sucrose. In a preferred embodiment, the percentage by weight of sucrolose ranges from about 0.1% to about 1%. In most preferred embodiments, the Sucrolose comprises from about 0.01% to about 1.0% by weight.

Numerous variations and modifications of the present invention are possible in light. of the above teachings. It is therefore to be understood that within the scope of the appended claims, the invention may be practiced otherwise than as specifically disclosed herein.

What is claimed as new and desired to be secured by Letters Patent of the United States is:

1. A composition comprising a prebiotic amount of one or more purified fructans, wherein at least one of said one or more purified fructans is predominately GFn, provided that the fructan component of the composition is not solely a mixture of 1-kestose, 1-nystose and 1^{f}-frutosylnystose.

2. A composition according to claim 1, wherein said at least one predominately GFn fructan has a degree of polymerization of greater than 4.

3. A composition according to claim 1, wherein said at least one predominately GFn fructan is a levan.

4. A composition according to claim 1, wherein said at least on predominately GFn fructan is a branched fructooligosaccharide.

5. A composition according to claim 1, wherein said at least one predominately GFn fructan is a mixture of short-chain fructans having an average degree of polymerization of 4.

6. A composition according to claim 1, comprising at least two or more predominately GFn fructans.

7. A composition comprising a prebiotic amount of one or more purified fructans, provided that said one or more purified fructans is not solely a mixture of predominately Fn FOS, and is not solely a mixture of 1-kestose, 1-nystose, and 1^{f}-fructosylnystose.

8. A composition comprising a prebiotic amount of one or more purified fructans, wherein at least one of said one or more purified fructans is predominately GFn and has a degree of polymerization greater than 4.

9. A composition comprising a prebiotic amount of one or more purified fructans, wherein the fructan in said composition is not solely one of Meioligo, Actilight, NutraFlora, Oligo-Sugar, Raftilose, Raftiline, or Fibruline.

10. A composition according to claim 1, wherein said purified fructan comprises less than about 5% glucose by weight.

11. A composition according to claim 3, wherein said levan is substantially unbranched.

12. A composition according to claim 3, wherein said levan is substantially branched.

13. A composition according to claim 1, wherein at least one of said one or more purified fructans is chosen from substantially branched inulins and substantially unbranched, predominately GFn inulins, wherein n is greater than 5.

14. A sweetener composition comprising:
i) one or more purified, predominately GFn fructans, wherein n ranges from 3 to 20; and
ii) a trichloro-glucose-fructose in an amount ranging from about 0.1% to about 1% by weight.

15. The sweetener composition of claim 14 wherein said trichloro-glucose-fructose ranges from about 0.01% to about 1.0% by weight.

16. A bifidogenic food additive comprising a prebiotic amount of one or more purified fructans, provided that said one or more purified fructans is not solely a mixture of predominately Fn FOS, and is not solely a mixture of 1-kestose, 1-nystose, and 1^{f}-fructosylnystose.

17. A method for promoting internal microbial balance, comprising contacting an internal body cavity with a composition comprising one or more purified fructans in an amount effective for promoting the proliferation of beneficial bacteria, wherein at least one of said one or more purified fructans is predominately GFn, provided that the fructan component of the composition is not solely a mixture of 1-kestose, 1-nystose, and 1^{f}-fructosylnystose.

18. A method according to claim 17 wherein said internal body cavity is intestinal or vaginal.

19. A method according to claim 18 wherein said internal body cavity is vaginal and said composition is in the form of a cream or emollient.

20. A method according to claim 17, wherein the beneficial bacteria is of the genus bifidus or lactobacillus.

21. A method according to claim 20, wherein the composition selectively promotes proliferation of said beneficial bacteria, not of pathogenic bacteria.

22. A method according to claim 20, wherein the pathogenic bacteria is E. coli or salmonella.

23. A low-calorie food additive comprising purified fructan, wherein said fructan is substantially branched, prebiotic, and comprises predominantly a mixture of GFn, where n ranges from 3 to about 20.
